(19) United States
(12) Patent Application Publication
(10) Pub. No.: US 2026/0069838 A1
(43) Pub. Date: Mar. 12, 2026

(54) CATHETER POSITIONING SYSTEMS

(71) Applicant: Vantis Vascular, Inc., San Jose, CA (US)

(72) Inventors: Tim A. FISCHELL, Kalamazoo, MI (US); Frank S. SALTIEL, Kalamazoo, MI (US)

(21) Appl. No.: 19/391,974

(22) Filed: Nov. 17, 2025

Related U.S. Application Data
(63) Continuation-in-part of application No. 18/953,702, filed on Nov. 20, 2024.
Application No. 18/953,702 is a continuation of application No. 18/191,400, filed on Mar. 28, 2023, now Pat. No. 12,186,509.
Application No. 18/191,400 is a continuation of application No. 16/793,120, filed on Feb. 18, 2020, now Pat. No. 11,642,500.
Application No. 16/793,120 is a continuation-in-part of application No. 16/132,878, filed on Sep. 17, 2018.
Application No. 16/132,878 is a continuation-in-part of application No. 15/899,603, filed on Feb. 20, 2018, now Pat. No. 11,491,313.

(51) Int. Cl. A61M 25/10; A61M 25/00; A61M 25/01; A61M 25/09

(52) U.S. Cl. CPC A61M 25/104; A61M 25/005; A61M 25/008; A61M 25/0102; A61M 2025/0081; A61M 25/09; A61M 2025/1079; A61M 2025/1081; A61M 2205/0266; A61M 2205/6081; A61M 2210/12

(57) ABSTRACT

Apparatuses for positioning a catheter may include an outer delivery sheath that may couple both centrally and distally to an inner tubular shaft to allow enhanced pushability of the outer delivery sheath. The system may include an outer delivery sheath having a middle junction, wherein the middle junction comprises a releasable lock, as well as an inner tubular shaft having a proximal end region, a distal end region, and an engagement protrusion extending radially from an outer surface of the inner tubular shaft, wherein the engagement protrusion is configured to engage the releasable lock when a distal end region of the inner tubular shaft extends distally out of a distal end of the outer delivery sheath. The engagement protrusion comprises a contact surface configured to drive advancement of the outer delivery sheath by pushing from the middle junction region of the outer delivery sheath.

### CLAIM OF PRIORITY

This application claims priority as a continuation-in-part of U.S. patent application Ser. No. 18/953,702, titled “INTRAVASCULAR DELIVERY SYSTEM AND METHOD FOR PERCUTANEOUS CORONARY INTERVENTION,” and filed on Nov. 20, 2024, which claims priority as continuation of U.S. patent application Ser. No. 18/191,400, titled “NTRAVASCULAR DELIVERY SYSTEM AND METHOD FOR PERCUTANEOUS CORONARY INTERVENTION,” filed on 28 Mar. 2023, which issued on Jan. 7, 2025 as U.S. Pat. No. 12,186,509, which is a continuation of U.S. patent application Ser. No. 16/793,120, titled “INTRAVASCULAR DELIVERY SYSTEM AND METHOD FOR PERCUTANEOUS CORONARY INTERVENTION,” filed on 18 Feb. 2020, issued as U.S. Pat. No. 11,642,500, which is a continuation-in-part (CIP) of U.S. patent application Ser. No. 16/132,878, titled “INTRAVASCULAR DELIVERY SYSTEM AND METHOD FOR PERCUTANEOUS CORONARY INTERVENTION,” filed on 17 Sep. 2018, currently pending, which is a continuation-in-part (CIP) of U.S. patent application Ser. No. 15/899,603, titled “GUIDE CATHETER EXTENSION SYSTEM WITH A DELIVERY MICRO-CATHETER CONFIGURED TO FACILITATE PERCUTANEOUS CORONARY INTERVENTION,” filed on 20 Feb. 2018, issued as U.S. Pat. No. 11,491,313.

### BACKGROUND

Coronary artery obstruction disease, or other disease in the peripheral vasculature, is often treated by a balloon angioplasty and/or stent placement. The advancement of revascularization devices, such as balloons or stent delivery systems, within blood vessels to a treatment site can be challenging to a physician where tortuosity and/or calcification of the vessels is found.

A coronary stent is a tube-shaped device placed in the coronary arteries that supply blood to the heart, to keep the arteries open for treatment of coronary heart disease which is used in a procedure commonly referred to as Percutaneous Coronary Intervention (PCI). Stents help to improve coronary artery blood flow, reduce chest pain and have been shown to improve survivability in the event of an acute myocardial infarction.

Treating a blocked coronary artery with a stent follows substantially the same steps as other angioplasty procedures, however there are important differences. The compressed stent mounted on a balloon significantly reduces the flexibility of the balloon and compromises its smooth advancement through the coronary artery. This can make it difficult or impossible to deliver the stent to a treatment site, and risks dislodgement of the un-deployed stent from its delivery balloon.

Intravascular imaging may be used to assess the lesion's thickness and hardness (calcification) which will affect the deliverability of the stent. A cardiologist uses this information to decide whether to treat the lesion with a stent, and if so, what kind and size of stent to be used. Stents, both bare metal and drug-eluting, are most often sold as a unit, with the stent in its collapsed (pre-expanded) form attached to the outside of a balloon catheter.

Physicians may perform “direct stenting,” where the stent is threaded through the vessel to the lesion and expanded. However, it is common to pre-dilate the blockage before delivering the stent in order to facilitate the stent delivery in more challenging lesions.

Pre-dilatation is accomplished by threading the lesion with an ordinary balloon catheter and expanding it to increase the lesion's diameter. A balloon catheter is a type of “soft” catheter with an inflatable balloon at its tip which is used during a catheterization procedure to enlarge a narrow opening or passage within the body. Subsequent to pre-dilatation, the pre-dilatation balloon is removed, and a stent catheter is threaded through the vessel to the lesion and is expanded which is left as a permanent implant to “scaffold” open the vessel at the lesion site.

Balloon catheters used in angioplasty have either over-the-wire (OTW) or rapid exchange (RX) design. The balloon catheter slides to the location over the guidewire which can be charged into the balloon catheter through a hub (in the over-the-wire modification) or through an RX port (for the rapid exchange modification of the balloon catheter). In the over-the-wire balloon catheter, a concentric lumen for passing the guidewire extends within the catheter from the proximal hub to the balloon, while in the rapid exchange (RX) balloon catheter, the lumen for the guidewire passage extends from the RX port inside the catheter to the balloon to permit the passage of the guidewire.

Revascularization devices usually use guiding (or guide) catheters for delivery of such devices to the site of treatment. The use of guide catheters alone to “back up” the advancement of the revascularization devices to the coronary arteries may be limited and challenging, especially when stents are placed using a radial access guiding catheter.

In order to facilitate the revascularization devices delivery to the site of interest, guide catheter extension systems have been designed and used during cardiac procedures.

For example, the guide extension system, such as “Guideliner™,” is produced by Teleflex. This guide extension system is described in U.S. Pat. No. 8,292,850, authored by Root, et al. Root, et al. (U.S. Pat. No. 8,292,850) and describes a coaxial guide catheter to be passed through a lumen of a guide catheter, for use with interventional cardiology devices that are insertable into a branch artery that branches off from a main artery.

The Root coaxial guide catheter is extended through the lumen of the guide catheter and beyond its distal end and inserted into the branch artery. Root uses the guide extension supported by a tapered inner catheter. The purpose of the inner catheter is to provide an atraumatic tip to avoid vessel injury, while advancing the guide extension into the proximal portion of a coronary vessel, in order to provide additional “backup” support for delivery of the stent or a balloon.

Another guide extension system, such as “Guidezilla™”, has been designed and manufactured by Boston Scientific. This guide extension system is described in U.S. Pat. No. 9,764,118, authored by Anderson, et al. Anderson's guide extension system uses a push member having a proximal portion having a proximal stiffness, a distal portion having a distal stiffness different from the proximal stiffness, and a transition portion which provides a smooth transition between the proximal and distal portions. A distal tubular member is attached to the push member and has an outer diameter larger than the outer diameter of the push member.

U.S. Patent Application Publication #2017/0028178, authored by Ho, describes a guide extension system using a slit catheter which is extendable upon insertion of a balloon or stent delivery system. Ho's guide extension also uses a rigid push rod to assist in delivery of the guide extension to the treatment site.

The systems, “Guideliner™” and “Guidezilla™”, as well as the Ho's system, support the concept of advancing the guide extension system through the guiding catheter, and partially down the coronary artery in order to achieve additional “back up” support to deliver balloon dilatation catheters and/or stent delivery catheters to the site of intended treatment.

The function of these guide extensions is to permit a closer approach to the lesion to provide additional support in crossing the lesion to be treated with an interventional device. However, despite the additional support, the lesion to be treated can still be difficult or nearly impossible to pass through with a pre-dilatation balloon catheter or a stent delivery system, due to fibrosis, calcification, prior stent struts in the lumen, and/or angulation at the lesion site.

One of the limitations of the currently used guide extension devices is that they use a relatively blunt and large caliber cylindrical distal end. Relatively high profile distal edges limit the deliverability of the guide extension in many cases and permit the advancement only to the proximal or mid portion of the coronary artery to be treated. Very rarely, if ever, can the guide extension be delivered to the actual lesion to be treated with angioplasty or stenting, even after balloon pre-dilatation of the lesion. These “blunt-ended” tubular guide extension devices may fail relatively frequently and may cause serious dissection complications. Published data demonstrate that “blunt-ended” tubular guide extension systems may fail it up to 20% of cases and cause serious coronary artery dissections in ˜3% of cases.

U.S. Patent Application Publication #2011/0301502, authored by Gill, describes a catheter with a longitudinal extension, allowing for the positioning device to be less in diameter than the stent delivery system. The Gill device, however, does not envision an inner catheter to permit easy and atraumatic crossing of the lesion to be treated. The Gill system acts merely as a covering for the stent delivery system, which can be removed after advancement of the stent delivery system due to the longitudinal extension.

Although the concept of a tapered piece inside a guide extension catheter is seen in the Root device, the prior art system uses a very short taper and does not envision the taper as an elongated integrated member of the entire system, nor does it envision that a pre-dilatation balloon can be attached to the tapered delivery micro-catheter to be delivered to the target treatment area. In addition, the prior art fails to envision a substantially “flush” interface between the inner catheter and the outer guide extension inside the vessel, or that the inner and outer catheter members would be reversibly fit or locked together to allow the entire system to be moved easily as one integral device.

Root or other prior art systems do not describe, anticipate or envision a balloon (and/or stent) delivery system, with a very low profile elongated tip which would be beneficial in attaining the coaxial delivery of the guide catheter extension/balloon system to, and beyond, a lesion of interest. Such an embodiment has never been commercialized, and the description of the tapered tip inner device was only meant as a mechanism for the proximal delivery of the blunt tip of the guide catheter extension out of the guiding catheter, but never as a mechanism for delivery of a balloon (and/or stent) to, and beyond, the target treatment area in a blood vessel, nor does it envision that the integral nature, and “flush” interconnection, of the inner and outer members would allow the passage of the outer delivery “sheath” member to cross the lesion of interest.

Thus, a device and method that would permit a delivery of the distal portion of the tubular guide extension system to, or ideally, beyond, the lesion to be treated, would have significant advantages over conventional guide extension devices, such as the “Guideliner™” (Teleflex), or the “Guidezilla™” (Boston Scientific), and others.

Neither of the conventional balloon catheters (over-the-wire or rapid exchange) is integrated with an outer delivery sheath, and neither of them uses a tapered delivery micro-catheter at the distal end of the catheter to which an interventional device (such as the balloon, or stent, etc.) would be secured for atraumatic advancement inside the blood vessel to, and beyond, the lesion site. In addition, none of the conventional balloon catheters is interconnected with an outer delivery sheath (guide catheter extension sub-system) via an interconnection mechanism actuated to permit integral motion of the conventional balloon catheter and the outer delivery sheath as a single unit, and deactuated to permit retraction of the balloon catheter from the outer delivery sheath, while preventing a forward displacement of the balloon catheter relative the outer delivery sheath.

It would be highly desirable, and efficient, to provide an intravascular delivery system which is capable of delivering an interventional device (for example, a pre-dilatation balloon) along with a guide catheter extension sub-system (such as an outer delivery sheath) to, and beyond, the lesion in a substantially atraumatic and convenient manner.

It also would be highly desirable to provide an intravascular delivery system which has an outer catheter and an inner catheter both featuring reinforced distal ends having a miniature tapered distal tip profile with a “seamless” distal interface to ensure an atraumatic crossability of the system to a lesion for treatment.

In addition, it would be desirable to facilitate percutaneous revascularization procedures by using a balloon attached to a coil reinforced tapered distal tip of the inner balloon catheter which is fitted within the outer delivery sheath of the outer catheter, where the inner balloon catheter is equipped with a distal elongated tapered coil-reinforced micro-catheter at the tapered distal tip to carry an interventional device (the pre-dilatation balloon, and/or stent) to, and past, the lesion to be treated. This would represent substantial improvement upon conventional guide catheter extension and pre-dilatation systems.

### SUMMARY OF THE DISCLOSURE

The present invention is directed to minimally invasive devices used for treatment within the human vasculature, such as, for example, coronary arteries, and, in particular, to a delivery system for percutaneous coronary intervention. These apparatuses may be adapted for intravascular balloon angioplasty and/or coronary artery stent delivery, enhanced by pre-dilatation guide catheter extension capabilities. In general, described herein are apparatuses (e.g., devices, system, etc.) for enhancing access to regions of the vasculature that may otherwise be difficult to access. In some cases these apparatuses may be systems, including catheter delivery systems.

The present invention is also directed to medical devices designed for atraumatic, convenient and fast delivery of various interventional devices, such as, for example, a pre-dilatation balloon, or stents, and replacement of catheters in coronary arteries (or other blood vessels) in a patient's body to facilitate percutaneous revascularization.

The subject invention further addresses an intravascular delivery system which has a miniature tapered soft distal tip which is capable of an exceptional deliverability of the subject interventional device which is superior to that of the conventional balloon angioplasty catheters along with a practicably atraumatic crossability thereof to a site of a lesion for treatment.

The present invention is also directed to an intravascular guide catheter extension/pre-dilatation system using an inner member (interventional device delivery catheter sub-system) positioned at a predetermined location internal an outer member (the outer delivery catheter sub-system), where the inner member is formed with a distal coil reinforced tapered portion interfacing with a slightly tapered distal end of the outer member. These are dimensioned to form a miniature profile and a substantially “seamless” transition at the interface between the distal ends of the outer member (outer catheter, equivalently referred to herein as an outer delivery sheath) and the inner member (inner catheter, equivalently referred to herein as an inner tubular shaft), at the transition point where the inner catheter's distal portion engages or enters the outer member. This structure is greatly beneficial for an atraumatic and smooth passage of the inner and outer member as a single unit along a diseased blood vessel.

Additionally, the present invention is directed to an intravascular guide catheter extension/pre-dilatation system configured with an outer catheter (member) and an inner catheter (member) displaceable inside and along the outer catheter where a distal tapered soft tip of the outer catheter is formed as an expandable flexible low durometer elastomeric member which, in its reduced configuration, has an inner diameter less than the outer diameter of the distal portion of the inner catheter at the area of engagement with the outer catheter. This arrangement attains a reversible elastic engagement between the outer and inner catheters at their distal ends, which ensures a return of the expanded distal end of the outer catheter to its reduced outer diameter when the inner catheter has been removed from the outer catheter, and reduces (or eliminates) “fish mouthing” at the distal junction of the outer and inner members as the system is advanced around the bends of a blood vessel.

Further, the present invention is directed to an intravascular guide catheter extension/pre-dilatation system configured with an outer and inner catheters displaceable one relative to another, where the proximal end of the outer catheter has an entry configuration providing an enhanced reinforcement, enhanced mid-shaft stent entry, prevention of stent embolization, increased flexibility, and flow rate improvement for a contrast infusion fluid.

Furthermore, the present invention is directed to an intravascular guide catheter extension/pre-dilatation system designed with a mid-shaft interconnection (locking) mechanism which is actuated/de-actuated by a physician to either (1) controllably engage the inner and outer members for the integral motion within a guide catheter along a guide wire, or (2) disengage the inner and outer catheters for retraction of the inner catheter from the outer member (catheter), as required by the intravascular procedure. The inner member may carry an interventional device (such as a pre-dilatation balloon member, or a stent) attached at its tapered coil reinforced distal end, and the locking mechanism provides a smooth, reversible engagement/disengagement procedure. This mid-shaft reversible locking also prevents any forward movement of the inner member relative to the outer member during system advancement or withdrawal and ensures that the position of the distal “seamless” transition of the inner and outer catheters remains essentially fixed in place axially during the movement of the subject system.

Additionally, the present invention is directed to an intravascular guide catheter extension/pre-dilatation system which is configured with a tapered coil reinforced shaft at its distal end for mounting and carrying a balloon member thereon and which provides a “seamless” entry and smooth deliverability of the balloon member integral with the outer catheter's coil reinforced delivery sheath to a desired treatment site.

The present invention further addresses an intravascular guide catheter extension/pre-dilatation system featuring a monorail micro-catheter embodiment with a Rapid Exchange (RX) feature for applications with short guidewires where the inner catheter's distal tapered soft end is configured with a coil-reinforced micro-catheter which provides additional kink resistance and “pushability” while still maintaining flexibility for navigating a tortuous vasculature.

It is therefore an object of the present invention to provide a medical device for intravascular applications that is capable of delivery of an interventional device (such as a balloon or a stent) to, and beyond, a coronary artery obstructive lesion in an efficient and minimally traumatic fashion.

It is another object of the present invention to provide an intravascular delivery system using a coaxial, highly flexible delivery catheter arrangement with an outer catheter and an inner catheter interfacing with each other at their distal ends in a “seamless” fashion with a miniature profile which is beneficial in attaining a “crossability” of the pre-dilatation balloon (or other interventional device) and enhances the efficient and safe distal delivery of a guide extension device.

It is an additional object of the subject invention to use a highly flexible coil-reinforced distal tapered elongated micro-catheter tip to deliver a pre-dilatation balloon (or another interventional device) to, and/or beyond, a target lesion in a diseased human coronary artery to be treated with angioplasty (or stenting).

It is a further object of the present invention to provide a guide catheter extension/pre-dilatation system using an outer catheter (outer delivery sheath sub-system) and an inner catheter (interventional device delivery sub-system) fitted and interchangeably connected within the outer sheath of the outer catheter, both deliverable to, or beyond, the lesion area of treatment within a blood vessel where the inner catheter has a delivery tapered micro-catheter at its distal end with the pre-dilatation balloon member (or another interventional device) attached thereto which slides along a guidewire in a substantially atraumatic manner.

It is a further object of the present invention to provide a guide catheter extension sub-system (outer member) integrated with the pre-dilatation balloon (or another interventional device) sub-system (inner member), in which the outer member and the inner member are coupled each to the other (via a locking mechanism) to be integrally (as a “whole system”) displaced along the guidewire to a lesion site. After the pre-dilatation procedure, the guide catheter extension sub-system (configured with an outer delivery sheath) is unlocked from the inner member and may be, if desired, advanced beyond the lesion. Subsequently, the inner member (interventional device delivery sub-system) may be withdrawn. The outer delivery sheath of the outer member may, if needed for the surgical procedure, remain in the guide catheter to enhance the deliverability of a stent (or other interventional device) to the lesion site inside the outer delivery sheath. The outer delivery sheath may be subsequently withdrawn after the stent (or other interventional device) has been delivered to the lesion and deployed for a definitive treatment.

Furthermore, it is an object of the present invention to provide a guide catheter extension/pre-dilatation system equipped with a “locking mechanism” operatively coupled between the inner member and the outer member (outer sheath) to provide integral passage of both the inner and outer members, as a single unit, for convenient and safe deliverability of the pre-dilatation balloon and the outer sheath to, and beyond, a treatment site.

It is a further object of the present invention to provide a guide extension system configured with the pre-dilatation balloon (or other interventional device) delivery catheter deliverable to the treatment site inside a vascular structure in an atraumatic manner to attain easy passage of the balloon (or other interventional device) and the guide extension system therethrough, thus expediting the cardiac procedure which permits percutaneous coronary intervention to be performed with a lower radiation dose exposure than would be achieved using conventional systems, with the added advantage of virtually no risk of stent embolization, or drug loss (with drug-eluting stents) from the stent delivery system.

A further object of the subject invention is to provide an intravascular guide catheter extension/pre-dilatation system configured with coaxial inner and outer catheters displaceable relative one another and enhanced by a coil reinforcement along their length, yet being increasingly flexible, and capable of attaining an improved contrast infusion flow rate and embolization prevention, where the tapered distal end of the outer catheter can be elastically stretched to form a strong contact with the distal portion of the inner catheter and nearly flush (smooth) outer surface at the interface between the inner catheter and the outer catheter.

The present system and method addresses an intravascular delivery system configured for controllable displacement along a guide wire in a blood vessel of interest. The subject system is formed with a proximal section, a distal section, and a middle section portion located between the proximal and middle sections. The current system includes an outer member formed by a flexible substantially cylindrically contoured elongated outer delivery sheath defining a sheath lumen having a proximal end and a distal end. The outer delivery sheath extends between the middle section and distal section and is configured with a tapered outer tip at the distal end of the sheath lumen. The tapered outer tip of the outer member at the distal end of the outer delivery sheath is configured with a wall extending in a cylindrical manner between a distal edge and a proximal edge of the tapered outer tip. The wall of the tapered outer tip has an inner diameter and an outer diameter. The inner and outer diameters of the wall of the tapered outer tip are gradually reduced in dimension from the proximal edge to the distal edge of the tapered outer tip. The proximal (wire or hypo-tube) element (pushing or pulling) connected to the outer member's tubular structure may be low profile and “flexible” (not “rigid”) to allow an enhanced conformability inside the guiding catheter and a lower profile than the rigid “pushing” elements in conventional guide extension catheters (as per Root). This is made possible due to the “pushability” of the “system as a whole”, attained via the locked and integral connection between the outer catheter (with its hypo-tube pushing/pulling element) and the inner catheter (guide extension tube).

The subject system further includes an inner member (inner catheter) having an elongated body defining an internal channel extending along its longitudinal axis. The inner member extends internally along the sheath lumen of the outer member (outer catheter) in a controllable relationship with the outer delivery sheath. The elongated body of the inner member has a tapered distal portion having an outer diameter and configured with a tapered delivery catheter having an elongated body of a predetermined length. The tapered delivery catheter of the inner member is displaceable beyond the distal end of the outer sheath. It is important that the inner diameter of the wall of the tapered outer tip of the outer member is less than the outer diameter of the tapered distal portion of the inner member at the area where the two elements form a distal junction.

An interconnection mechanism is operatively coupled between the inner and outer members and is controllably actuated to operate the guide catheter extension/pre-dilatation sub-system in an engaged or disengaged mode of operation. In the engaged mode of operation, the inner and outer members of the guide catheter extension sub-system are engaged for a controllable common displacement along the guide wire. This also allows the enhanced “pushability” of the subject system (with the outer member connected and locked to the inner member) even with the connected pusher (pushing/pulling element) of the outer member having a miniature profile and being flexible (as flexible or more flexible than the outer tubular sheath of the outer catheter). In the disengaged mode of operation, the inner and outer members are disengaged for retraction of the inner member from the outer member subsequent to the pre-dilatation treatment, or stent, delivery.

The distal portion of the inner member interfaces, at the outer surface thereof, with an inner surface of the tapered outer tip of the sheath lumen. A dimensional transition between the outer diameter of the outer tip of the sheath lumen and the outer diameter of the distal tip of the inner member forms a substantially flush interface transition therebetween.

The tapered outer tip of the outer member has an elastically expandable configuration. At the proximal end thereof (also referred to herein a mid-shaft portion of the outer member), the outer sheath is configured with an entrance opening exceeding in its circumference the circumference of the tubular body of the outer sheath. In some embodiments, the entrance opening at the proximal end of the outer sheath is funnel shaped.

The outer sheath is preferably reinforced along its length. The outer member comprises a distal soft tip encapsulating material enveloping the reinforced sheath of the outer member at its distal end. The distal soft tip encapsulating material is a flexible low durometer elastomeric material having a gradient durometer value increasing from the distal end towards the proximal end of the sheath.

The outer member also includes a distal lubricous liner sandwiched between an outer surface of the outer sheath and an inner surface of the distal soft tip encapsulating material.

The delivery catheter is preferably a micro catheter. The micro-catheter is formed of a flexible material and may have a differential flexibility along its length, wherein the flexibility of the micro-catheter increases towards its distal end.

A balloon member is attached to the tapered distal portion of the inner member in proximity to the tapered delivery micro-catheter; and an inflation lumen extends within the inner member between the proximal section and the balloon member at the distal section to provide a fluid passage between an external balloon inflation system and the balloon member. The balloon member may assume an inflated configuration or a deflated configuration. In the deflated configuration, the balloon member is displaced in the blood vessel. The balloon member is controllably transformed into the inflated configuration subsequent to being positioned at least in alignment with the treatment site for the pre-dilatation procedure.

The elongated body of the inner member and the micro-catheter are coil reinforced along their length.

An outer catheter's pusher/puller element configured with a flattened portion at its distal end is secured to the proximal end of the outer sheath of the outer catheter. Preferably, the outer member's pusher/puller is configured with a channel extending along its length in fluid communication with the sheath lumen in order to prevent embolization. This proximal (pushing and pulling) element connected to the outer catheter's outer sheath tubular structure may be low profile and “flexible” (not “rigid”) to allow better conformability inside the guiding catheter and a lower profile than the rigid “pushing” elements in conventional guide extension catheters (such as Root).

The interconnection mechanism may include a snap-fit locking mechanism configured with a proximal coupler disposed at the proximal end of the sheath of the outer member (catheter) and a cooperating element disposed at the outer surface of the elongated body of the inner member (catheter). The proximal coupler may include a distal solid ring and a mid split ring positioned a predetermined distance from the solid ring, while the cooperating member includes a member selected from a group including a mid-shift lock ring, square annular ring, snap-fit cage and other like members. The cooperating member is affixed to the outer surface of the elongated body of the inner member. When the cooperating member is engaged and locked in a snap-fit fashion between the distal solid ring and the mid split ring, a locking engagement between the outer and inner members is attained. The proximal pusher/puller element of the outer catheter and the coupler may be made from a memory metal (such as, for example, nitinol), so as to prevent deformation during the antegrade or the retrograde movement of the outer member and to prevent any deformation of the mid-shaft coupler (also referred to herein as a proximal coupler) during the stent or other device passage through the mid-shaft portion of the outer catheter.

The proximal coupler further includes a proximal beveled split ring at its proximal end which reinforces the funnel like proximal entrance of the outer member and prevents damage or permanent deformation of the funnel shaped proximal entrance caused by displacement of the inner member or a stent delivery system in the funnel entrance. The coupler and mid-shaft entry may have an entrance opening (or a “mouth”) the circumference of which is larger than the circumference of the outer member's flexible tubular outer sheath structure.

The subject intravascular system further includes a guide wire advanceable in a blood vessel of interest to at least a treatment site, wherein the guide catheter extension sub-system is configured for controllable displacement along the guide wire. In one of the subject system's embodiments, an elastic outer jacket envelopes the inner member, at least at its proximal end, and the inner member's pusher/puller along at least its distal end. The proximal end of the inner member is connected to the pusher/puller by fusing the elastic outer jacket to the length of the proximal end of the inner member and supporting the inner member's pusher/puller snuggly in the elastic outer jacket.

The pushing-pulling element (or its outer jacket) of the outer catheter may be color coated to have a distinguished color to differentiate it from the pushing/pulling element of the inner catheter, as well as from the usual gray or silver color of a coronary guidewire. Alternatively, the elastic outer jacket of the inner member may be color coated to distinguish the inner member's pusher/puller from the color(s) of other elements in the subject system for the surgeon's convenience.

For example, described herein are apparatuses (e.g., systems and devices) including any of these features. In particular, described herein are systems including: an outer delivery sheath (also referred to herein in some examples as an outer member or outer catheter) having a proximal region, a distal region, and a middle junction (e.g., middle junction region) between the proximal and distal regions, wherein the middle junction comprises a releasable lock; and an inner tubular shaft (also referred to herein as an inner member or inner micro-catheter) having a proximal end region, a distal end region, and an engagement protrusion extending radially from an outer surface of the inner tubular shaft, wherein the engagement protrusion is configured to engage the releasable lock when a distal end region of the inner tubular shaft extends distally out of a distal end of the outer delivery sheath; wherein the engagement protrusion comprises a contact surface configured to drive advancement of the outer delivery sheath by pushing from the middle junction of the outer delivery sheath.

The inner tubular shaft may comprise a contact region proximal to a distal end of the inner tubular shaft that is configured to form a substantially flush interface with the distal end of the outer delivery sheath when the engagement protrusion engages the releasable lock.

Any of these systems may include a connector configured to releasably connect the proximal end region of the outer delivery sheath to the proximal end region of the inner tubular shaft when the engagement protrusion engages the releasable lock. The connector may be a snap connector.

The releasable lock may comprise a friction lock. The releasable lock may comprise an engagement slot on the outer delivery sheath configured to receive the engagement protrusion from the inner tubular shaft. The releasable lock may comprise a lock ring configured to elastically expand.

The engagement protrusion may comprise a ring.

The proximal region of the outer delivery sheath may comprise a wire. The distal region of the outer delivery sheath may comprises a tubular distal region. The inner tubular shaft may comprise a rapid exchange notch having an opening into a lumen extending from the rapid exchange notch to a distal end of the inner tubular shaft.

The inner tubular shaft may be configured to extend more than 1.5 cm distally out of a distal end of the outer delivery sheath when the engagement protrusion engages the releasable lock.

For example, a system may include: an outer delivery sheath having a proximal wire, a tubular distal region, and a middle junction between the proximal wire and the tubular distal region, wherein the middle junction comprises a releasable lock; and an inner tubular shaft having a proximal end region, a distal end region, and an engagement protrusion extending radially from an outer surface of the inner tubular shaft, wherein the engagement protrusion is configured to lock with the releasable lock when a distal end region of the inner tubular shaft extends distally out of a distal end of the tubular outer delivery sheath; wherein the distal end of the outer delivery sheath is configured to from a flush interface with a contact region of the inner tubular shaft that is proximal to a distal end of the inner tubular shaft when the engagement protrusion engages the releasable lock; further wherein the engagement protrusion comprises a contact surface configured to drive advancement of the outer delivery sheath by pushing the middle junction.

The system may include a snap connector configured to releasably secure a proximal end region of the outer delivery sheath together with the proximal end region of the inner tubular shaft when the engagement protrusion is locked with the releasable lock. The releasable lock may comprises a friction lock. The releasable lock may comprise an engagement slot on the outer delivery sheath configured to receive the engagement protrusion from the inner tubular shaft. The releasable lock may comprise a lock ring.

The engagement protrusion may comprises a ring.

The inner tubular shaft may comprise a rapid exchange notch having an opening into a lumen extending from the rapid exchange notch to a distal end of the inner tubular shaft.

The inner tubular shaft may be configured to extend more than 1.5 cm distally out of a distal end of the outer delivery sheath when the engagement protrusion is locked with the releasable lock.

For example, a system may include: an outer delivery sheath having a proximal wire, a tubular distal region, and a middle junction between the proximal wire and the tubular distal region forming an opening into a lumen of the tubular distal region, wherein the middle junction comprises a releasable engagement region; an inner tubular shaft having a proximal end region, a distal end region, and an engagement protrusion extending radially from an outer surface of the inner tubular shaft, wherein the engagement protrusion is configured to engage with the releasable engagement region when a distal end region of the inner tubular shaft extends distally out of a distal end of the tubular outer delivery sheath; a connector configured to releasably secure a proximal end region of the outer delivery sheath together with the proximal end region of the inner tubular shaft when the engagement protrusion is engaged with the releasable engagement region; further wherein the engagement protrusion comprises a contact surface configured to drive advancement of the outer delivery sheath by pushing the middle junction.

In general, any of the apparatuses and methods described herein may be configured as systems in which the distal end region of the outer member (e.g., the outer delivery sheath or outer catheter), may include a tapered distal end region that is configured to seal around a surface of an interface region (e.g., “landing zone”) of the inner tubular shaft (e.g., inner member or inner microcatheter) just proximal to the distal end of the inner tubular shaft when the middle junction of the outer delivery sheath is engaged with the projection of the inner tubular shaft so that the outer surface of the outer delivery sheath distal end is flush (and tapers down to) the outer surface of the interface region of the inner tubular shaft. This may allow the both the middle regions and the distal end regions of the inner and outer members to be engaged (with the distal end regions sliding but still flush and/or sealed) as the system is navigated through tortious anatomy, which may also enhancing pushing and delivery of the otherwise very narrow-diameter outer delivery sheath.

For example, the system may include: an outer delivery sheath having a tubular distal region, a proximal wire, and a middle junction between the tubular distal region and the proximal wire, wherein the middle junction comprises a releasable lock, and wherein the tubular distal region has a tapered distal tip formed of a stretchable material; and an inner tubular shaft having a distal end region, a proximal end region, and a middle region comprising an engagement protrusion configured to engage the releasable lock, wherein the distal end region has an interface region that is proximal to a distal end of the inner tubular shaft, and wherein the tapered distal tip of the outer delivery sheath has an inner diameter that is less than or equal to an outer diameter of the interface region; further wherein the outer delivery sheath and inner tubular shaft are sized and configured so that when the engagement protrusion is engaged with the releasable lock, an outer surface of the tapered distal tip is substantially flush with a surface of the interface region.

The engagement protrusion may extend radially from an outer surface of the inner tubular shaft.

In any of these systems the tapered distal tip may have a smaller inner diameter than a region proximal to the tapered distal tip. The stretchable material may be a polymeric material, such as a very flexible, low durometer elastomeric Pebax material. Thus, in general the distal tip region may be elastically deformable so as to snugly seal against the outer surface of the interface region. The interface region may extend proximal to a tapered distal tip at a distal end of the inner tubular shaft. The outer diameter of the interface region may be relatively constant; in some examples the interface region is between 5 mm and 4 cm long (e.g., between 1 cm and 5 cm, between 1 cm and 4 cm, etc.); this length is helpful, as the tapered distal tip may move distally and proximally over the interface region of the inner tubular shaft as the region between the coupled middle regions of the outer and inner members may change relative length with bending, while maintaining the flush transition between the inner and outer members.

In any of these apparatuses, the interface region may be flanked by tapered regions proximally and distally to the interface region; e.g., the region distal and proximal may taper down from the larger outer diameter of the interface region, compared to the more proximal and/or more distal regions. As mentioned, the distal tip of the inner tubular shaft may also be tapered down. For example, the interface region may have an outer diameter that is wider than a region of the inner tubular shaft proximal to the interface region and distal to the interface region.

In any of these apparatuses the engagement protrusion may be any appropriate protrusion, including but not limited to a ring.

The releasable lock may comprise an expandable ring.

The middle junction of the outer delivery sheath may comprise a funnel shaped opening into a lumen of the outer delivery sheath.

For example, a system may include: an outer delivery sheath having a tubular distal region, a proximal wire, and a middle junction between the tubular distal region and the proximal wire, wherein the middle junction comprises a tapered proximal opening into a lumen of the tubular distal region and a releasable lock, and wherein the tubular distal region has a tapered distal tip formed of a stretchable material; and an inner tubular shaft having a distal end region configured to be inserted into the lumen of the outer delivery sheath, a proximal end region, and a middle region comprising an engagement protrusion configured to engage the releasable lock, wherein the distal end region has an interface region that is proximal to a distal end of the inner tubular shaft, and wherein the tapered distal tip of the outer delivery sheath is configured so that an outer surface of the tapered distal tip is substantially flush with a surface of the interface region when the engagement protrusion is engaged with the releasable lock.

The engagement protrusion may extend radially from an outer surface of the inner tubular shaft. The tapered distal tip of the outer delivery sheath may have a smaller inner diameter than a region proximal to the tapered distal tip of the outer delivery sheath.

A system may include: an outer delivery sheath having a tubular distal region, a proximal wire, and a middle junction between the tubular distal region and the proximal wire, wherein the middle junction comprises a funnel-shaped opening into a lumen of the tubular distal region and a releasable lock, and wherein the tubular distal region has a tapered distal tip formed of a stretchable material; and an inner tubular shaft having a distal end region configured to be inserted into the lumen of the outer delivery sheath, a proximal end region, and a middle region comprising an engagement protrusion configured to engage the releasable lock, wherein the distal end region has an interface region that is proximal to a distal end of the inner tubular shaft, and wherein the tapered distal tip of the outer delivery sheath has an inner diameter that is less than or equal to an outer diameter of the interface region and is configured so that when the engagement protrusion is engaged with the releasable lock, an outer surface of the tapered distal tip is substantially flush with a surface of the interface region.

As described in greater detail herein, these systems may be used to perform an intervascular procedure on a patient. For example, any of these methods may include: advancing a guide catheter over a guidewire within a blood vessel; driving an inner tubular shaft distally to advance an outer delivery sheath over the guidewire and distal to the guide catheter by pushing the outer delivery sheath from a medial region of the outer delivery sheath, wherein the medial region of the outer delivery sheath is engaged with a protrusion extending from a medial region of the inner tubular shaft; maintaining a distal end of the outer delivery sheath substantially flush with a contact region of the inner tubular shaft that is proximal to the distal end of the inner tubular shaft as the inner tubular shaft and outer delivery sheath are advanced together distally as an integral unit; disengaging the inner tubular shaft from the outer delivery sheath by withdrawing the inner tubular shaft proximally; and performing a procedure through the outer delivery sheath.

Any of these methods may include positioning the distal end of the outer delivery sheath at or beyond a treatment site before disengaging the inner tubular shaft. Any of these methods may include engaging the inner tubular shaft with the outer delivery sheath by advancing the inner tubular shaft distally over the guidewire and through the guide catheter and outer delivery sheath until an engagement protrusion extending radially from an outer surface of the inner tubular shaft engages with a releasable engagement in the medial region of the outer delivery sheath while the distal end of the inner tubular shaft extends distal to the distal end of the outer delivery sheath.

Any of these methods may include releasably locking the medial region of the outer delivery sheath to the protrusion extending from the medial region of the inner tubular shaft, and/or engaging a proximal end region of the outer delivery sheath to proximal region of the inner tubular shaft.

The methods may include removing the inner tubular shaft from the patient, e.g., before performing the procedure. For example, any of these methods may include disengaging the inner tubular shaft by unlocking the medial region of the outer delivery sheath from the inner tubular shaft by pulling the inner tubular shaft proximally relative to the outer delivery sheath to unlock the medial region of the outer delivery sheath from the inner tubular shaft.

In general, performing the procedure may comprise delivering a treatment device through the outer delivery sheath. Delivering the treatment device through the outer delivery sheath may include delivering a stent. Delivering the treatment device through the outer delivery sheath may include delivering a balloon catheter.

In any of these methods, pushing the outer delivery sheath from the medial region of the outer delivery sheath may comprise pushing a distal bearing surface of a ring forming the protrusion extending from the medial region of the inner tubular shaft against a releasable lock of the outer delivery sheath in the medial region of the outer delivery sheath.

Maintaining the distal end of the outer delivery sheath substantially flush with the contact region of the inner tubular shaft may comprise expanding a tapered opening of the outer delivery sheath against the contact region. The contact region (e.g., ‘landing zone’) may be configured to accommodate relative changes in the lengths of the inner tubular shaft (e.g., microcatheter) relative to the outer delivery sheath (e.g., outer catheter).

For example, a method of performing an intervascular procedure on a patient may include: advancing a guide catheter over a guidewire within a blood vessel; advancing an inner tubular shaft distally over the guidewire to advance an outer delivery sheath distally out of the guide catheter by pushing the outer delivery sheath from a medial region of the outer delivery sheath, wherein the medial region of the outer delivery sheath is engaged with the inner tubular shaft at both the medial region of the outer delivery sheath and a distal end of the outer delivery sheath, wherein the medial region of the outer delivery sheath is coupled to a protrusion extending from a medial region of the inner tubular shaft, further wherein the distal end of the outer delivery sheath is held flush against an outer contact region proximal to a distal end of the inner tubular shaft; maintaining the distal end of the outer delivery sheath substantially flush with the outer contact region of the inner tubular shaft as the inner tubular shaft and outer delivery sheath are advanced together distally as an integral unit; disengaging the inner tubular shaft from the outer delivery sheath by withdrawing the inner tubular shaft proximally; and performing a procedure in the blood vessel through the outer delivery sheath.

Any of these methods may include engaging the protrusion extending from a medial region of the inner tubular shaft with the medial region of the outer delivery sheath by advancing the inner tubular shaft distally over the guidewire until the protrusion engages with a releasable engagement in the medial region of the outer delivery sheath and the distal end of the outer delivery sheath is substantially flush with the outer contact region of the inner tubular shaft. Any of these methods may include releasably locking the medial region of the outer delivery sheath to the protrusion.

Any of these methods may include engaging a proximal end region of the outer delivery sheath to proximal region of the inner tubular shaft.

As mentioned, these methods may include removing the inner tubular shaft from the patient after disengaging the inner tubular shaft from the outer delivery sheath. For example, disengaging the inner tubular shaft may comprise unlocking the medial region of the outer delivery sheath from the inner tubular shaft by pulling the inner tubular shaft proximally relative to the outer delivery sheath to unlock the medial region of the outer delivery sheath from the inner tubular shaft.

The procedure may include delivering a treatment device through the outer delivery sheath, for example, delivering the treatment device through the outer delivery sheath comprises delivering a stent. In any of these methods delivering the treatment device through the outer delivery sheath may comprise delivering a balloon catheter.

For example, a method of performing an intervascular procedure on a patient may include: advancing a guide catheter over a guidewire within a blood vessel; advancing an inner tubular shaft distally to releasably lock a medial region of the inner tubular shaft to a releasable lock in a medial region of an outer delivery sheath; advancing the inner tubular shaft and the outer delivery sheath together distally over the guidewire as an integral unit by advancing the inner tubular shaft distally over the guidewire to push the outer delivery sheath from the medial region of the outer delivery sheath where the releasable lock is engaged with the medial region of the inner tubular shaft; maintaining a distal end of the outer delivery sheath substantially flush with an outer contact region proximal to a distal end of the inner tubular shaft as the inner tubular shaft and outer delivery sheath are advanced together distally; disengaging the inner tubular shaft from the outer delivery sheath by withdrawing the inner tubular shaft proximally; and performing a procedure in the blood vessel through the outer delivery sheath.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 schematically represents the subject guide catheter extension/pre-dilatation system advanced to the target site within a coronary artery;

FIGS. 2A-2C schematically describe the subject guide catheter extension/pre-dilatation system, where FIG. 2A shows the assembled inner and outer catheters, FIG. 2B details the inner catheter, and FIG. 2C details the middle section of the subject system;

FIG. 3A-3D are representative of the middle section of the subject inner catheter with FIG. 3A showing a longitudinal section of the inflation lumen hypo-tube interconnected with the inflation lumen distal shaft in the inner catheter, FIG. 3B detailing a longitudinal section of the skived portion of the inflation lumen hypo-tube, FIG. 3C showing a longitudinal section of the inner catheter depicting an RX guide wire (GW) port formed in the inflation lumen distal shaft, and FIG. 3D showing an isometric view of the RX port portion of the inner catheter shown in FIG. 3C;

FIG. 4 shows a longitudinal section of the inner catheter detailing the distal end of the inflation hypo-tube at the junction with the inflation lumen distal shaft;

FIGS. 5A-5C depict the distal section of the subject system with FIG. 5A depicting the inflated balloon member, FIG. 5B presenting the deflated balloon member, and FIG. 5C detailing the inflation lumen/balloon junction;

FIGS. 6A-6B depict the longitudinal section of the distal section of the subject inner catheter detailing the balloon's 3 mm distal and proximal tapers (FIG. 6A) and the balloon's 6 mm distal and proximal tapers (FIG. 6B);

FIG. 7 depicts the distal tip of the outer catheter;

FIGS. 8A-8B detail the interfacing of the inner and outer catheters at their distal ends, where FIG. 8A is representative of the inner catheter's tapered distal end, and FIG. 8B depicts on a somewhat enlarged scale, the connection point between the inner and outer catheters;

FIGS. 9A-9D are representative of alternative embodiments of the elastically stretchable distal tip of the outer catheter configured with an expandable split ring (FIG. 9A), expandable tip scaffold (FIG. 9B), and slits (FIGS. 9C-9D);

FIGS. 10A-10G depict a side view (FIGS. 10A, 10B, 10D, 10F), and an isometric view (FIGS. 10C, 10E, 10G) of the alternative embodiments of the proximal portion of the subject outer catheter;

FIGS. 11A-11C detail the design of the coupler at the proximal end of the outer catheter, where FIG. 11A depicts an isometric view of the flattened hypo-tube pusher, FIG. 11B is an isometric view of the proximal end of the outer catheter, and FIG. 11C depicts a side view of the coupler at the proximal end of the outer catheter featuring a snap-fit lock mechanism;

FIGS. 12A-12C depict an alternative embodiment of the proximal portion of the subject outer catheter, with FIG. 12A being an isometric view of the proximal coupler, FIG. 12B being an isometric view of the encapsulated proximal coupler, and FIG. 12C being a side view of the encapsulated proximal coupler;

FIGS. 13A-13B illustrate an isometric view (FIG. 13A) at a side view (FIG. 13B) of yet another embodiment of the proximal coupler at the outer catheter's proximal end;

FIGS. 14A-14B depict welded rings embodiment of the proximal entry of the outer catheter, with FIG. 14A showing the welded rings coupler, and FIG. 14B showing the encapsulated welded rings coupler;

FIGS. 15A-15D depict additional alternative embodiments of the proximal coupler of the outer catheter, with FIG. 15A-15B being a side view and isometric view, respectively, of the circularly contoured funnel fenestrations, and FIGS. 15B-15D being a side view and isometric view, respectively, of the triangular funnel fenestrations;

FIGS. 16A-16C are representative of the hypo-tube pusher flush lumen concept, with FIG. 16A depicting an isometric view of the proximal coupler of the outer catheter coupled with the pusher, FIG. 16B being a sectional isometric view of FIG. 16A depicting a flow channel in the pusher, and FIG. 16C showing a procedure of injecting a flushing fluid between the inner and outer catheters;

FIGS. 17A-17C depict the subject mid-shaft annular round ring lock mechanism, where FIG. 17A shows the “lock disengaged” mode of operation, FIG. 17B illustrates the “lock engaged” mode of operation, and FIG. 17C is representative of the annular round ring in the subject lock mechanism;

FIGS. 18A-18B detail the subject annular round ring lock mechanism shown in FIGS. 17A-17B. where FIG. 18A depicts the proximal coupler configured with the lock pocket for engagement with the annular round ring (of FIG. 17C), and FIG. 18B is a longitudinal cross-section of the inner/outer catheters locked one to another;

FIGS. 19A-19C depict the alternative subject embodiment of the lock mechanism, featuring a mid-shaft “square” annular ring, with FIG. 19A illustrating the inner catheter equipped with the ring shaped lock mechanism, FIG. 19B illustrating the inner catheter's ring snapped in the outer catheter's proximal coupler, and FIG. 19C depicting a cross-sectional view of the square annular ring;

FIGS. 20A-20C depict the alternative “snap-fit cage” lock mechanism, with FIG. 20A showing the inner catheter with the welded cage lock, FIG. 20B showing the inner catheter's welded cage lock snapped in the outer catheter's proximal coupler, and FIG. 20C being an isometric view of the welded cage element;

FIG. 21 is a side view of another embodiment of the outer catheter's proximal coupler featuring two locking slots;

FIG. 22A-22B are representative of a monorail micro-catheter embodiment of the subject system with FIG. 22A depicting an isometric view of the monorail micro-catheter embodiment, and FIG. 22B showing a side view taken along Lines A-A;

FIG. 23A-23C detail the subject monorail micro-catheter embodiment with FIG. 23A showing the isometric view of the proximal portion of the inner catheter connected with the inner catheter hypo-tube pusher, FIG. 23B detailing the proximal end of the pusher on a somewhat enlarged scale, and FIG. 23C being a side view of the proximal portion of the inner catheter connected with the hypo-tube pusher; and

FIGS. 24A and 24B depict an isometric view and a side view, respectively, of the coil reinforced balloon catheter embodiment of the subject system.

### DETAILED DESCRIPTION

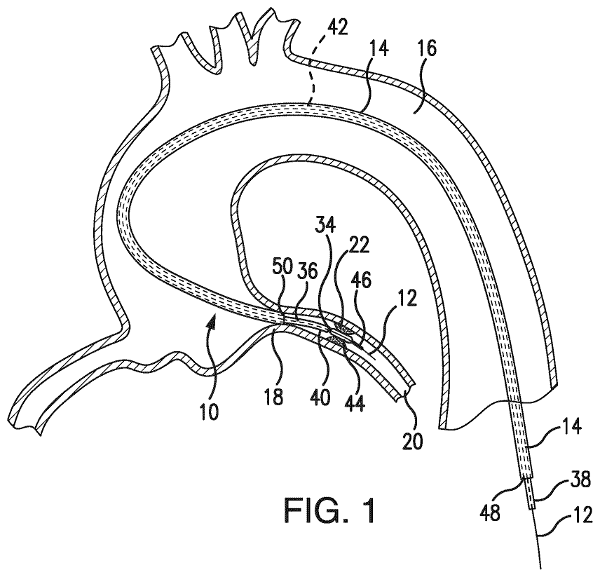

Depicted in FIGS. 1-24B, is the subject intravascular delivery system 10 which includes a guide catheter extension sub-system (also referred to herein as an outer catheter or an outer member) and an interventional device delivery sub-system (also referred to herein as an inner catheter or an inner member) cooperating under control of a surgeon during a cardiac procedure. Although the interventional device delivery sub-system may be used for delivery of various cardiac interventional devices, in one of the implementations, as an example only, but not to limit the scope of the subject invention to this particular embodiment, the subject interventional device delivery sub-system will be further described as adapted for delivery of a balloon member for performing the pre-dilatation procedure.

In the exemplary embodiment described herein, the subject system 10 may be referred to herein as a guide catheter extension/pre-dilatation system which may be used for cardiac procedures in conjunction with a guide wire 12 and a guide catheter 14. As shown in FIG. 1, at the initial stage of the cardiac procedure, the guidewire (GW) 12 is moved by a surgeon into the blood vessel 16. The guide catheter 14 is advanced through the blood vessel 16 (such as, for example, the aorta) along the guide wire 12 to a position adjacent to the ostium 18 of the coronary artery 20. The guidewire 12 may be used during the cardiac procedure to guide the guide catheter 14, and, subsequently, the subject guide catheter extension/pre-dilatation system 10 (inside the guide catheter 14) may be extended within the artery 20 toward a target location 22, as will be detailed in following paragraphs.

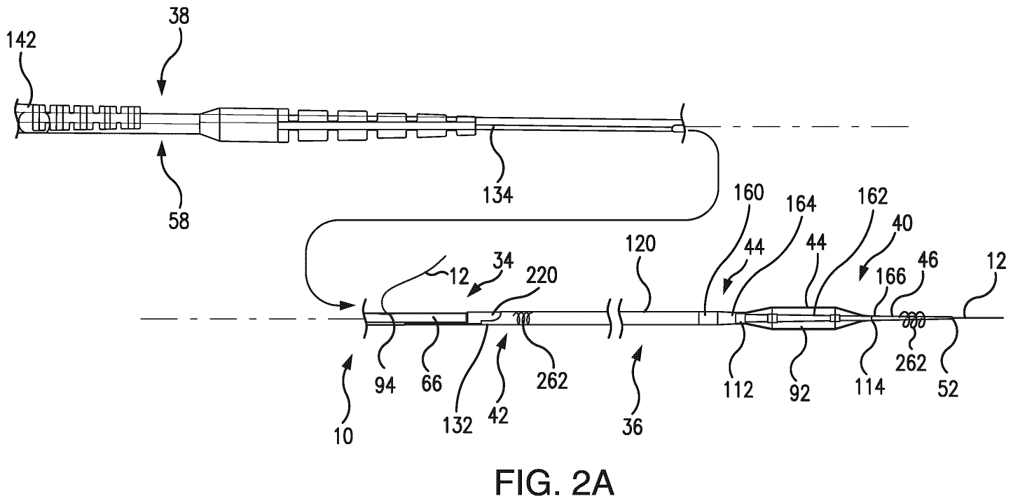

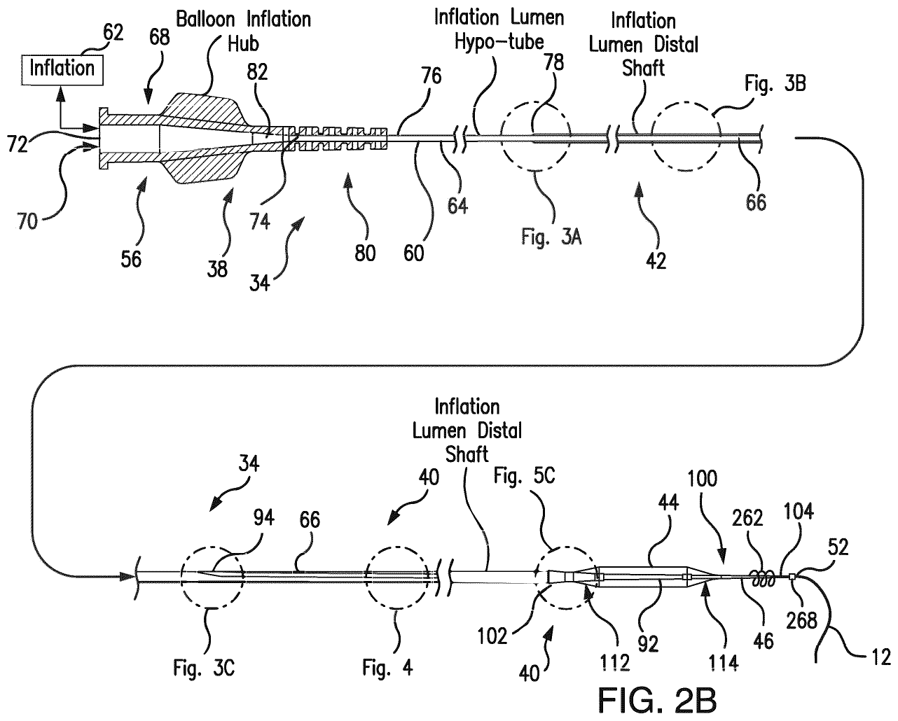

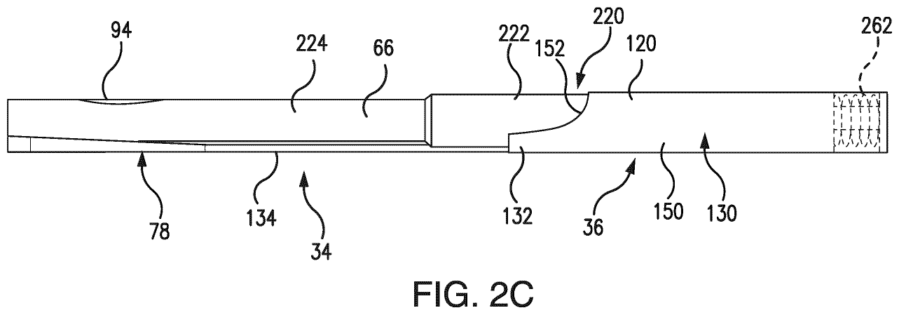

As shown in FIGS. 2A-2C, the subject guide catheter extension/pre-dilatation system 10 includes a balloon catheter sub-system 34 (also referred to herein as an inner catheter, inner member, or a pre-dilatation sub-assembly) and a guide catheter extension sub-system 36 (also referred to herein as an outer delivery sheath or outer catheter). The inner catheter 34 interacts with the outer catheter 36 and can be engaged with or disengaged from the outer catheter 36, as required by the cardiac procedure.

The subject system 10 includes a proximal section 38, a distal section 40, and a middle section 42 extending between and interconnecting the proximal and distal sections 38, 40. A pre-dilatation balloon member 44 is carried at the distal section 40 of the inner catheter 34. The distal section 40 of the inner catheter 34 also may be configured with an elongated tapered inner tubular shaft 46 (e.g., inner member, also referred to as a micro-catheter), as will be detailed in the following paragraphs.

The subject guide extension/pre-dilatation system 10, as shown in FIG. 1, is extended within a lumen (internal channel) 48 of the guide catheter 14. In order to reliably reach the target location 22, and, in some cases, pass beyond the target location 22, the subject guide extension/pre-dilatation system 10, is advanced through the guide catheter 14 beyond a distal end 50 of the guide catheter 14 deep into the coronary artery 20. The subject system 10, by extending beyond the distal end 50 of the guide catheter 14, provides an adequate reachability of the pre-dilatation balloon 44 to the target location 22, and, by extending beyond the ostium 18 of the coronary artery 20, stabilizes the positioning of the guide catheter 14 and allows for an improved accessibility for the subject system 10 into the coronary artery 20 and to the target site 22.

As shown in FIGS. 1, 2A-2B, 3C-3D, 4, 5A-5C, and 6A, the guide wire 12 extends internal the guide catheter extension/pre-dilatation system 10, and exits the system 10 with the distal end of the GW 12 beyond the outermost end 52 of the distal section 40 and with the proximal end of the GW 12 at the middle section 42.

In operation, the inner catheter 34 and the outer catheter 36 are coupled one to another to be advanced (as a single unit) along the guide wire 12 inside the guide catheter 14 positioned within the blood vessel 16, and extend beyond the distal end 50 of the guide catheter 14 to reach the target lesion site 22. Once the subject balloon catheter sub-system (inner member) 34 reaches the lesion site 22, and the balloon member 44 is positioned in alignment with the lesion site 22, the intended pre-dilatation procedure may be performed. Once the pre-dilatation has been performed, the outer catheter (also referred to herein as outer member) 36 may be advanced across the lesion as an integral unit with the inner catheter (also referred to herein as an inner member) 34, with subsequent disengagement of the inner catheter 34 from the outer catheter 36 for withdrawal of the inner catheter from the outer catheter.

Alternatively, after the pre-dilatation procedure has been performed, the inner catheter 34 may be disengaged from the outer catheter 36, while the outer catheter 36 is advanced across the dilated lesion. In addition, the outer catheter 36 may be left in proximity to the lesion after the pre-dilatation has been performed and the inner catheter 34 has been removed.

In any case scenario, the outer member (catheter) 36 remaining in proximity to the pre-dilated lesion may be used for delivery of a stent inside the outer member (catheter) 36 to the lesion site. The outer member 36 is removed from the guide catheter 14 once the stent is installed (deployed) at the lesion site.

As will be presented in further paragraphs, in the subject system, the inner catheter 34 is prevented from forward displacement inside the outer catheter 36. Exclusively a backward or removal displacement of the inner member 34 relative to the outer member 36 is permitted to support retraction of the inner member from the outer member subsequent to the pre-dilatation of the lesion.

Referring to FIGS. 2A-2C, the proximal section 38 of the subject guide extension/pre-dilatation system 10 is represented by a balloon inflation hub 56 (best depicted in FIG. 2B) of the inner member 34 and a proximal end 58 of an outer member 36.

Referring to FIGS. 2B, 3A-3D, 4, and 5C, the inner member (also referred to herein intermittently as the balloon catheter sub-system or pre-dilatation balloon delivery sub-system) 34 is configured with an internal inflation channel 60 extending between the inflation hub 56 and the pre-dilatation balloon member 44. The internal inflation channel 60 serves as a passage for inflation air between a balloon inflation system 62 (shown schematically in FIG. 2B) and the balloon member 44 for the controlled inflation/deflation of the balloon member 44 as prescribed by the cardiac procedure.

The internal inflation channel 60 is formed by an inflation lumen hypo-tube 64 and an inflation lumen distal shaft 66 overlappingly interconnected each to the other in a fluidly sealed manner.

The inflation hub 56 located at the proximal end 68 of the inner member 34 is configured with an internal cone-shaped channel 70 which is connected by its proximal opening 72 to the balloon inflation system 62 (as schematically shown in FIG. 2B).

The balloon inflation system 62 may be a manual or an automatic system. In a preferred automatic embodiment, the balloon inflation system 62 includes an electronic sub-system, a pneumatic sub-system and control software with a corresponding user interface. The electronic sub-system, under control of the control software, supplies power to solenoid pressure valves (which are fluidly coupled to the balloon inflation hub 56) to control the pressurizing/depressurizing of the balloon member 44 with fluid or air flow.

As shown in FIG. 2B, the internal cone-shaped channel 70 of the balloon inflation hub 56 is configured with a distal opening 74 which is coupled to the inflation lumen hypo-tube 64. The proximal end of the inflation lumen hypo-tube 64 is coupled to the distal opening 74 of the internal cone-shaped channel 70 of the balloon inflation hub 56 in a fluidly sealed fashion to support passage of the inflation air between the balloon member 44 at the inflation system 62.

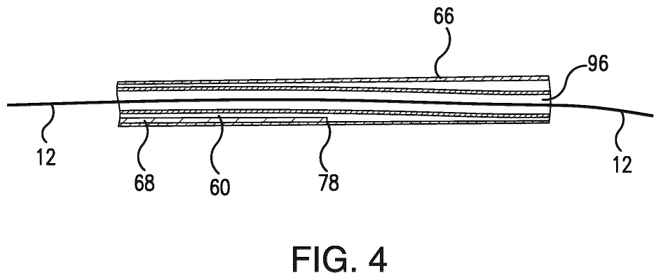

The inflation lumen hypo-tube 64 extends through the length of the proximal section 38 and a portion of the middle section 42 of the subject system 10 and terminates with its distal end 78 at the distal section 40, as shown in FIGS. 2B and 4.

As shown in FIG. 2B, a flexible serrated member 80 is provided at the proximal end 76 of the inflation lumen hypo-tube 64 which is coupled to the distal end 82 of the balloon inflation hub 56. The serrated flexible member 80 supports the proximal end 76 of the inflation lumen hypo-tube 64 and provides a flexible bending of the structure when manipulated by a surgeon.

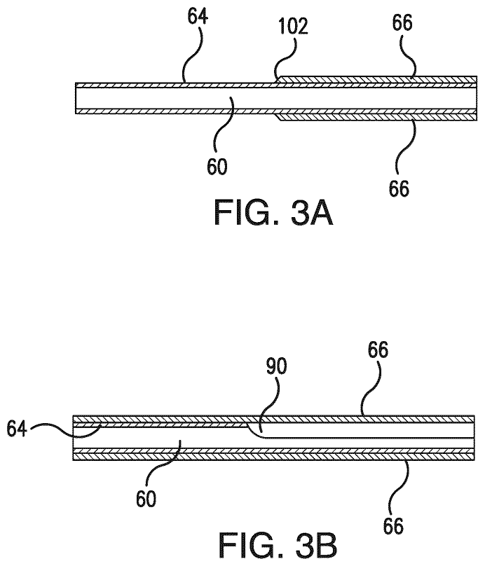

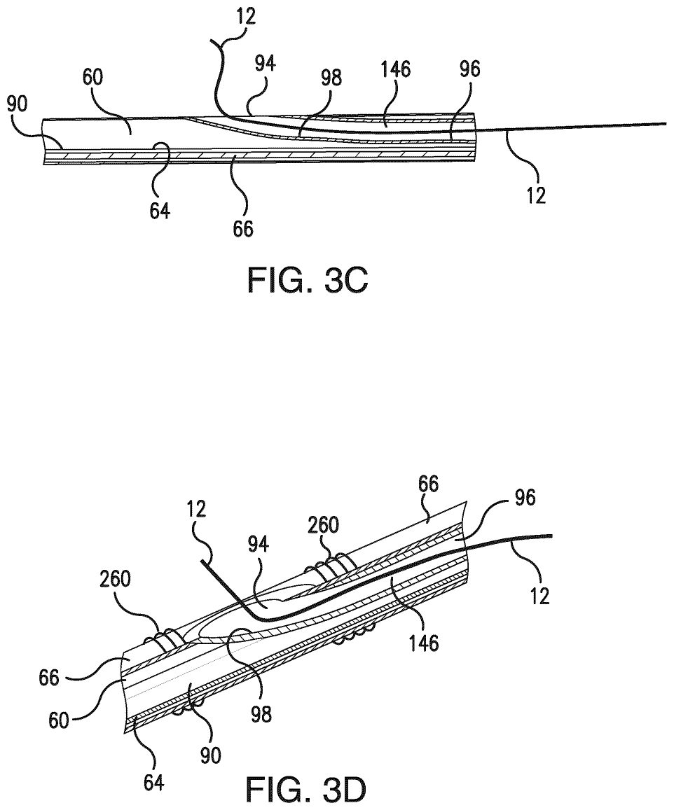

As shown in FIGS. 2A-2C, 3A-3D, 4 and 5C, the inflation lumen distal shaft 66 extends between the proximal section 38 along the middle section 42 and ends at the distal section 40. FIG. 3A details the junction between the inflation lumen hypo-tube 64 and the inflation lumen distal shaft 66. The inflation lumen hypo-tube 64 does not extend all the way through the inner member 34 but terminates at its distal end 78 (as shown in FIGS. 2B and 4).

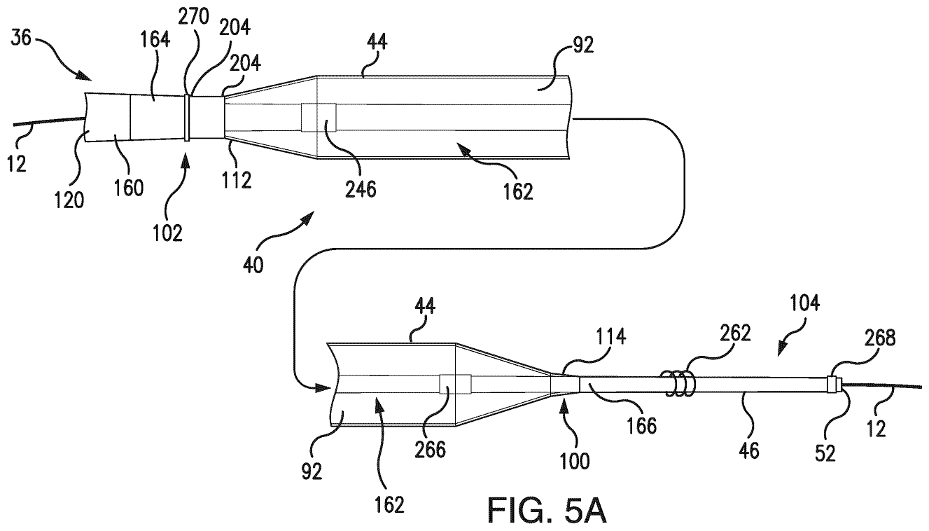

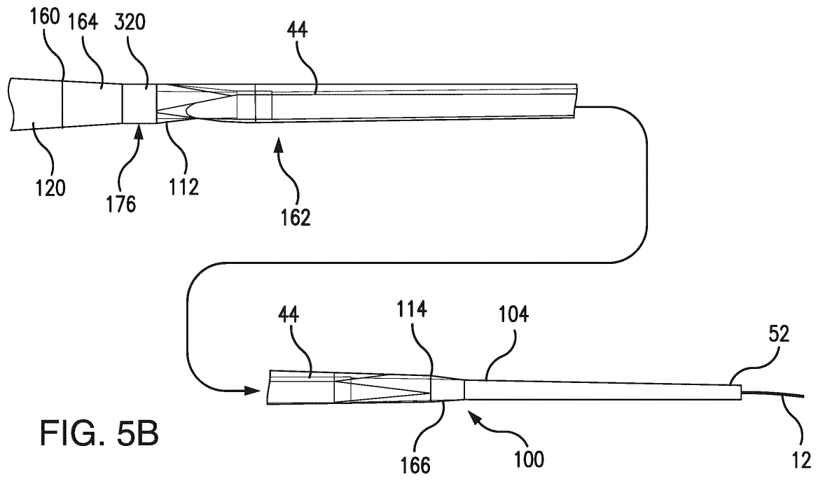

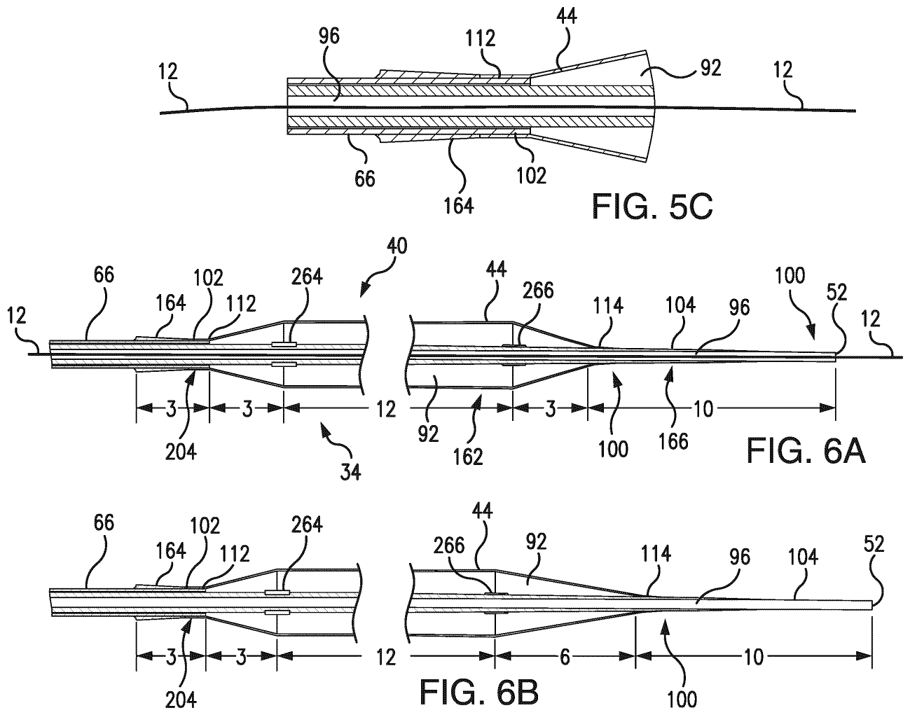

Referring to FIGS. 3B-3D, the inflation lumen hypo-tube 64 has a skived distal portion 90 which is coaxially enveloped by the wall of the inflation lumen distal shaft 66 so that the inflation lumen hypo-tube 64, in conjunction with the inflation lumen distal shaft 66, provide a sealed fluid communication between the balloon inflation system 62 and the internal chamber 92 of the balloon member 44, as shown in FIGS. 5A-5C, for controlled inflation/deflation of the balloon member 44 as required by the cardiac procedure.

FIGS. 2B and 3C-3D illustrate that the inflation lumen distal shaft 66 is configured with a rapid exchange (RX) guidewire (GW) port 94 at which a GW lumen 96 begins with its proximal end 98. The GW lumen 96 extends between the RX GW port 94 inside the inflation lumen distal shaft 66 through the entire length of the distal section 40 of the inner catheter 34. The GW lumen 96 forms an internal channel with the proximal end 98 corresponding to the RX GW port 94 and a distal end 100 corresponding to the outermost distal end 52 of the distal section 40 of the inner member 34. As shown in FIGS. 6A-6B, at the distal section 40, the GW lumen 96 extends beyond the distal end 102 of the inflation lumen distal shaft 66. The distal end 100 of the GW lumen 96 constitutes a gradually tapered portion 104 which may be in the form of a delivery micro-catheter 46.

Referring to FIGS. 2A-2B, 5A-5C, 6A-6B, and 24A-24B, the inner catheter (also referred to herein as a balloon catheter sub-system) 34 is configured with a tapered distal portion (also intermittently referred to herein as tapered distal tip) 162 at the distal section 40. The tapered distal portion 162 is equipped with the pre-dilatation balloon member 44 which is secured onto the tapered distal portion 162 in close proximity to the micro-catheter 46. The pre-dilatation balloon member 44 is secured to the inner member's tapered distal portion (tip) 162 for supporting the pre-dilatation/stenting procedure, as required for the cardiac treatment of a patient.

The balloon member 44 has a proximal portion 112 and a distal portion 114. The balloon member 44 is attached (secured) at the distal section 40 in proximity to the delivery micro-catheter 46 with its proximal portion 112 coupled to the distal end 102 of the inflation lumen distal shaft 66, and with the distal portion 114 of the balloon 44 to the outer surface of the micro-catheter 46.

As shown in FIGS. 5A-5C, the pre-dilatation balloon 44 is attached, with its proximal portion 112, to the proximal portion 204 of the distal tip 162 in bordering juxtaposition with the outer tip 164 of the sheath 120, and, with its distal portion 114, to the distal end 166 of the distal portion (tip) 162 of the inner member 34.

The balloon member 44 may intermittently assume deflated (folded) and inflated (expanded) configurations. The deflated (folded) configuration is used during insertion and/or withdrawal of the subject system relative to the blood vessel. The balloon is inflated (expanded) when in place (at the target site 22) to widen the blood vessel and compress the plaque for pre-dilatation procedure, or for the stenting procedure (when a stent is delivered to the treatment site on a balloon). When inflated, the balloon 44 assumes the inflated/open configuration shown in FIGS. 2A-2B, 5A, 5C, 6A-6B, and 24A-24B for pre-dilatation of the diseased blood vessel. When deflated, the balloon member 44 assumes the deflated configuration shown in FIG. 5B.

The balloon 44 may have a smooth surface, or a “chocolate” configuration. The “chocolate” balloon catheter is an over-the-wire balloon dilatation catheter with a braided shaft and an atraumatic tapered tip. The balloon, when expanded, is constrained by a nitinol structure that creates small “pillows” and grooves in the balloon.

Referring now to FIGS. 2A, 2C, 5A-5C, 7, 8A-8B 9A, 9C-9D, 10A-10G, 11C, 12B-12C, 13A-13B, 14B, 15A-15D, 16A-16B, 17A-17B, 18A-18B, 19B, 20A-20B, 21, and 24A-24B, the outer catheter (also referred to as the guide catheter extension sub-system) 36 is formed with a cylindrical outer delivery sheath 120 having an internal channel 122 extending internally there along. A coupler mechanism 130 is formed at the proximal end 132 of the cylindrical sheath 120 in encircling relationship therewith.

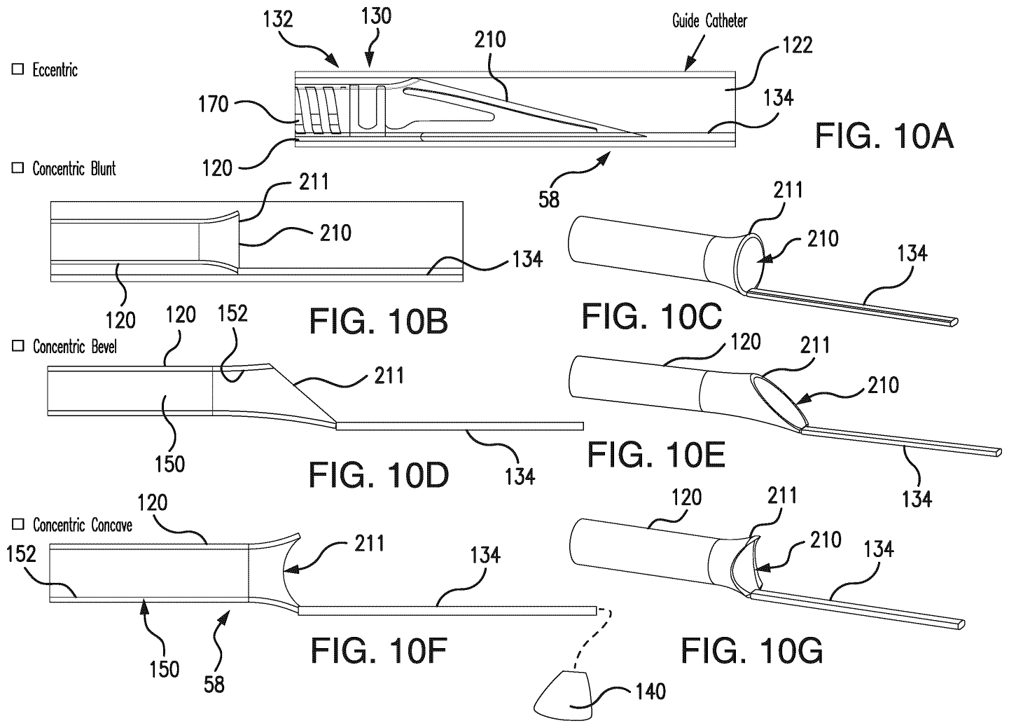

At the proximal end 58, the outer catheter 36 includes an outer member pusher (also referred to herein as a pusher/puller) 134, which, as shown in FIGS. 10B-10G, 11A-11C, 12A-12C, 13A-13B, 14A-14B, 15A-15D, 16A-16B, 17A-17B, 18A-18B, 19B, and 22, in one embodiment, may be a solid wire which may have a round wire proximal section 136, and a flattened distal portion 138 which may be welded or otherwise fixedly attached to the proximal end 132 of the sheath 130. In another embodiment, the pushing and pulling element 134 may be configured with a hypo-tube.

Alternatively, a round pusher wire can be welded to a flat wire which, in its turn, is welded or otherwise fixedly secured to the proximal end 132 of the sheath 120.

In still another alternative embodiment of the outer member 36, a round wire may be welded or otherwise fixedly secured to two flat wires, which in their turn, are welded or otherwise fixedly secured to the proximal end 132 of the sheath 120.

The flattened profile of the pusher wire portion is welded to the proximal coupler 130 of the outer sheath 120 so that when the inner member 34 is inserted in the outer member (catheter) 36, the pusher wire does not create an obstacle for the rotational or longitudinal motion of the inner catheter 34 inside the proximal coupler 130 and the sheath 120 of the outer member 36, as required by the procedure. The proximal pushing-pulling element 134 advances with or withdraws the outer tubular sheath 120 and is preferably flexible (not rigid). The pusher/puller 134 may be flexible (not rigid) with the flexibility along its longitudinal axis being comparable or exceeding the flexibility of the tubular outer delivery sheath 120 of the outer catheter 36.

The outer catheter's pusher 134 may be equipped, at the proximal end thereof, with a proximal handle 140, shown in FIG. 10F, for convenience of a surgeon performing the coronary intervention procedure for manipulation of the outer member 36 in order to position the outer delivery sheath 120 along with the balloon delivery sub-system 34, at the desired location relative to the lesion 22 in the diseased blood vessel.

The proximal (wire or hypo-tube configured) pushing/pulling element 134 connected to the outer member's tubular structure 120, by having a miniature profile and being flexible (not “rigid”), attains an enhanced conformability inside the guiding catheter and a lower profile than the rigid “pushing” elements in conventional guide extension catheters (as per Root). This is made possible due to the “pushability” of the “system as a whole”, attained via the locked integral connection between the outer catheter (with its hypo-tube pushing element) and the inner catheter (guide extension tube).

In addition, the inner catheter (inner member) 34 may be equipped with an inner member's pusher (also referred to herein as a pusher/puller) 142 (shown in FIG. 2A) which may be attached to the inflation hub 56 to facilitate the withdrawal of the inner member 34 from the outer member 36 as required by the coronary intervention procedure, as well as for controlling engagement/disengagement therebetween, for various stages of the cardiac procedure. The inner member's pusher/puller 142 may be formed with an inner member pusher/puller's handle for convenience of a surgeon performing the procedure.

The handles of the inner and outer members' pushers may be configured with a mechanism, e.g., connector, (also detailed in the U.S. patent application Ser. No. 15/899,603 which is hereby incorporated by reference) which permits an additional releasable locking of the inner and outer members one to the other to enhance the integral cooperation thereof in an engaged mode of operation.

The inner member 34 (inner tubular shaft) may be either of the over-the-wire configuration or of the RX configuration. In one of the embodiments detailed herein, the guide wire 12 extends through the RX GW port 94 made at the proximal end of the tubular inflation lumen distal shaft 66 into and along the internal channel 146 of the GW lumen 96, as shown in FIGS. 3C-3D, and 4. At the distal section 40 of the subject system 10, the guidewire 12 extends in the GW lumen along the delivery tapered micro-catheter 46 (at the tapered portion 104) and exits at the distal ends 100 of the GW lumen 96 at the outermost end 52 of the inner member 34, as shown in FIGS. 2A-2B, 5A-5B, and 6A-6B.

The distal outer delivery sheath 120 of the outer member 36 is fabricated with a flexible cylindrically shaped tubular body 150 extending substantially the length of the middle section 42 of the subject system 10. By manipulating the outer member pusher 134, a surgeon actuates the integral advancement of the outer delivery sheath 120 and the inner member 34 along the guide catheter 14. When the pre-dilatation procedure has been performed (as will be detailed in further paragraphs), the surgeon controls a required linear backward displacement of the inner member 34 with regard to the sheath 120 of the outer member 36 by manipulating the outer member pusher 134 and/or the inner member pusher 142.

In general, it has been found that advancing the outer delivery sheath by pushing on the proximal wire (“pusher”) 134 is not always sufficient, particularly in tortious regions of the anatomy. However, as described in greater detail here, engaging the outer delivery sheath with an inner tubular shaft (e.g., inner member, in some examples a micro-catheter) at an intermediate region (e.g., a middle junction or section, also referred to herein as a middle junction region) 42 in a pushable engagement, particularly when the distal end region of the inner tubular shaft is seamlessly engaged with the distal end opening of the outer delivery sheath, the outer delivery sheath may be safely, quickly and significantly more easily advanced distally through even tortious region that are otherwise impassible pushing proximally from just the proximal end of the outer delivery sheath. Without being bound by theory, this may be attributed to driving the outer delivery sheath from more distal regions, e.g., from this middle junction region and the distal end region of the outer delivery sheath, which may distribute the force in a manner that reduces buckling and compression of the outer delivery sheath, permitting it to be softer (e.g., more flexible) and therefore less traumatic on the vessel.

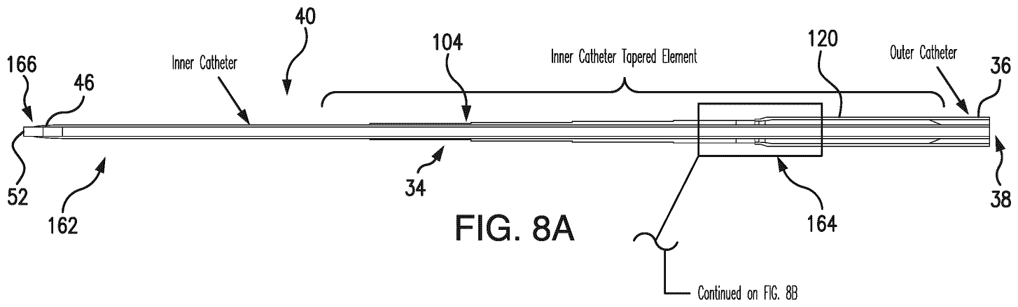

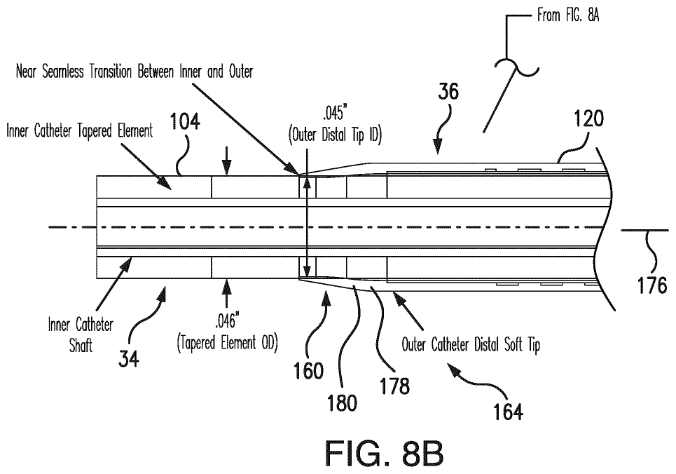

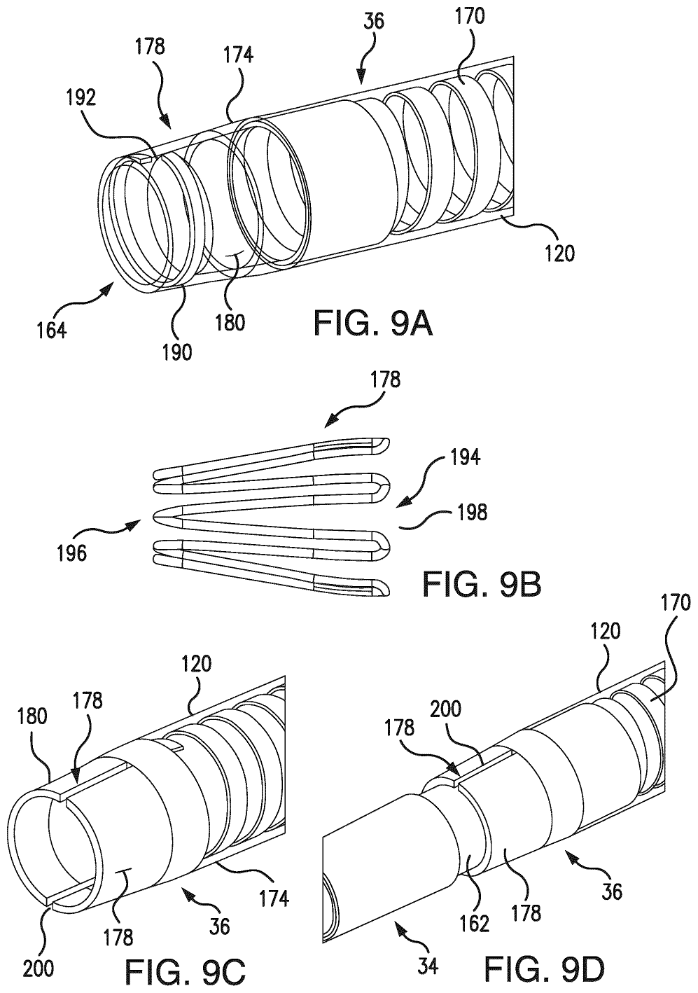

The interface between the outer tip 164 of the sheath 120 and the distal tip 162 of the inner member 34, as shown in FIGS. 8A-8B and 9A-9D, facilitates displacement of the distal tip 162 of the inner member 34 relative to the outer tip 164 of the sheath 120 and basically facilitates displacement of the distal tip 162 relative to the outer tip 164 of the sheath 120 as required by the cardiac procedure.

The distal end 160, as well as the outer tip 164 of the sheath 120, is formed of a flexible material which permits a simplified retraction of the distal tip 162 of the inner member 34 therethrough. The flat wire helical coil may be used for the distal end 160 and the outer tip 164 of the sheath 120. The flexible material may be a stretchable material (e.g., an elastomeric material) such as a low-durometer material (e.g., a durometer of less than 50 ShoreA, less than 45 ShoreA, less than 40 ShoreA, less than 35 ShoreA, less than 30 ShoreA, less than 25 ShoreA, less than 20 ShoreA, less than 15 ShoreA, less than 10 ShoreA, etc.).

At its proximal end 132, the sheath 120 of the outer catheter 36, is configured with an entrance “opening” (or a “mouth”) 210 the circumference of which exceeds the circumference of the outer member flexible tubular sheath 120, as shown in FIGS. 10A-10G. The entrance 210 (also referred to herein as a “mouth”) into the internal channel 122 of the sheath 120 may be configured in various modifications. For example, as shown in FIG. 10A, the entrance 210 has a funnel shape 211 with an eccentric opening (as shown in FIG. 10A), or contoured with a concentric blunt (as shown in FIGS. 10B-10C), or with a concentric bevel (as shown in FIGS. 10D-10E), or alternatively, with the concentric concave contour (as shown in FIGS. 10F-10G). The pusher 134 is affixed at a predetermined point of the funnel shape outer catheter's proximal entry 210.

As shown in FIGS. 2A, 2C, 7, and 8A-8B, the outer delivery sheath 120 of the outer catheter 36 extends between its proximal end 132 at the middle section 42 and its distal end 160 at the distal section 40 of the subject system 10. At the distal section 40 of the subject guide catheter extension/pre-dilatation system 10, the inner member 34 is configured with a tapered configuration 104 having a distal tapered portion (also referred to herein as a distal tapered tip) 162 which may be formed with the micro-catheter 46, as shown in FIGS. 2A-2B, 5A-5B, 6A-6B, 8A, 22A-22B, and 24A-24B. The micro-catheter 46 is an elongated thin member with the length in a cm range, for example, 1-3 cm. The micro-catheter 46 has a tapered cone-contoured configuration with the diameter not exceeding 1 mm at its distal end 52. The micro-catheter 46 may be formed integrally with the tapered distal tip 162 of the inner member 34.

As shown in FIGS. 2A, 5A-5C, 7, and 8A-8B, at the distal end 160, the outer delivery sheath 120 is formed with an outer tip 164 which has a tapered cone-contoured configuration which may be interconnected with the distal tip 162 of the inner member 34. The outer tip 164 of the outer member 36 provides a smooth distal taper transition between the distal end 160 of the sheath 120 and the distal section 40.

In FIGS. 2A, 5A-5B, 6A-6B, 8A-8B, 22A-22B, and 24A-24B, the distal tip 162 of the inner catheter 34 is shown to have a tapered configuration which changes gradually from the point of interconnection with the outer tip 164 of the sheath 120 to the distal end 166 of the distal tip 162. The micro-catheter 46 extends from the distal end 166 of the distal tapered portion 162 of the inner member 34 (the length of about 1-3 cm) in an integral connection therewith and terminates in the outermost distal end 52.

The subject guide catheter extension/pre-dilatation system 10 may be configured with a differential in micro-catheter flexibility with greater flexibility in the distal portion, by either changing the durometer of the plastic (polymeric) components from the outer delivery sheath's proximal portion to its distal portion (i.e., a higher durometer in the proximal portion when taken with respect to the distal portion), and/or changing the winding frequency (pitch) of the helical coil of wire in the micro-catheter 46 in the direction from the proximal portion to distal portion, such that the distal portion of the micro-catheter 46 is more flexible and trackable than the proximal portion of the micro-catheter delivery device, with a substantially lower profile and is more flexible than the distal portion of the guide catheter extension sub-system (outer delivery sheath).

The system 10 may also include wires that have radio-opacity such that the balloon member 44, micro-catheter 46, and the outer delivery sheath 120 are easily visualized using fluoroscopy. It is envisioned that the distal tip 162 (as shown in FIGS. 5A, 6A-6B) is provided with radio-opaque markers 264, 266 in proximity to the proximal portion 112 and the distal portion 114 of the balloon 44. The radio-markers 264, 266 permit the surgeon (operator) to visualize positioning of the balloon member 44 relative to the lesion location 22.

In addition, the outermost distal tip 52 of the micro-catheter delivery portion 46 and the tip 160 of the sheath 120 may have one or more radio-opaque markers 268, 270 (shown in FIGS. 2B and 5A) in order to permit the surgeon to distinguish between the radio-markers, which is particularly important as the obstructive lesion is passed by the micro-catheter, and the balloon member carried in proximity to the micro-catheter is held in place.

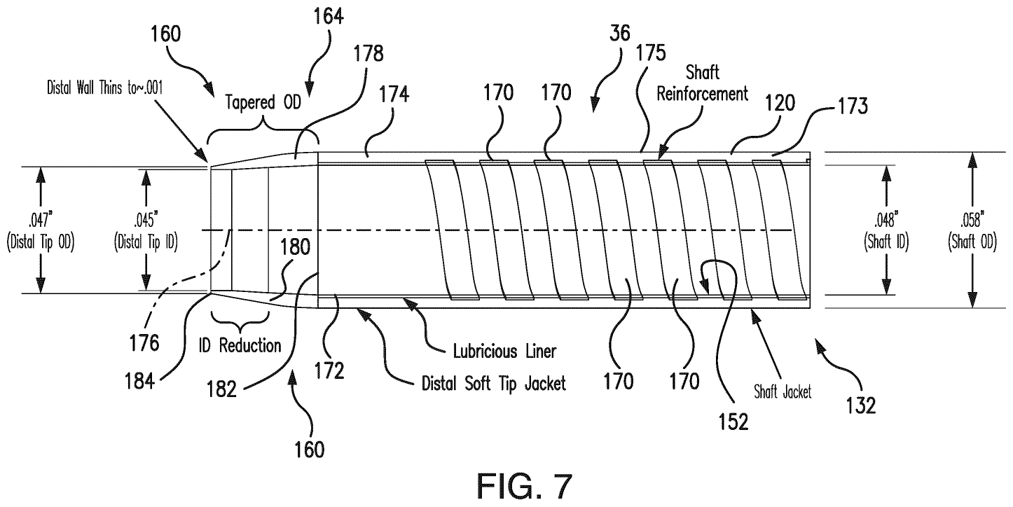

As detailed in FIG. 7, in one embodiment thereof, the outer catheter 36 is configured with a system of catheter shaft coil reinforcement 170 disposed on (or embedded in) the internal surface 152 of the sheath 120. Preferably, a lubricious liner 172 is positioned inside the shaft 120. The shaft reinforcement coil 170 may be installed inside the shaft 120 in contact with the lubricious liner 172, i.e., in encircling relationship with the surface of a lubricious liner 172 which covers the inner surface 152 of the shaft 120. A distal soft tip jacket 174 is affixed at the distal end of the outer catheter shaft 120 along the longitudinal axis 176 of the outer catheter 36.

The distal soft tip jacket 174 may be glued to the shaft 120 at the end 175 (as shown in FIG. 7) or may cover some length of the outer surface 173 of the shaft 120.

The distal soft tip jacket 174 extends at the distal end 160 of the shaft 120 beyond the coil reinforcement 170 and the lubricious liner 172, and terminates in the tapered portion 178, which has a distal edge 184 and a proximal edge 182.

The lubricious liner 172 may be formed from the PTFE material. The distal soft tip jacket 172 may be formed of a very flexible low durometer elastomeric Pebax material which transitions into high durometers along the longitudinal axis 176 towards the proximal end 132 of the sheath 120.

As shown in FIGS. 7 and 8A-8B, in one of the preferred embodiments, the internal diameter of the sheath 120 at its inner surface 152 is approximately 0.048″, while the outer diameter of the shaft 120 at its outer surface 173 is 0.058″. The internal diameter of the tapered portion 178 of the outer catheter 36 at the distal edge 184 is 0.045″, while the outer diameter of the tapered portion 178 at its distal edge 184 is 0.047″. The gradient between the outer diameter (0.058″) of the sheath 120 and the outer diameter of the taper 178 (0.047″) define the outer surface tapering, while the gradient between the inner diameter (0.048″) of the sheath 120 at the inner diameter (0.045″) of the taper 178 at its distal edge 184 define the tapering of its inner surface. The distal wall 180 of the tapered portion 178 has a thickness reduction from the interface 182 (between the sheath 120 and the tapered portion 178) to the outermost edge 184 of the tapered portion 178 of the distal soft tip jacket 174.

As shown in FIG. 7 in conjunction with FIGS. 8A-8B, the outer diameter of the inner catheter's tapered element 104 has the outer diameter approximately 0.046″ which is approximately 0.001″ larger than the outer catheter's distal tip internal diameter (0.045″) at the outermost distal edge 184. This difference between the outer diameter of the tapered element 104 of the inner catheter 34 and the inner diameter at the distal edge 184 of the outer catheter's outer tip 164 results in stretching of the distal soft tip jacket 174 at the tapered portion 178 thereof when interfering with the inner catheter's tapered element 104. Such arrangement provides for a near seamless transition between the distal tip of the inner catheter 34 and the distal tip of the outer catheter 36, as well as a miniature profile of the distal end due to the squeezing of the distal tip of the inner catheter 34 by the tapered element 178 of the outer catheter 36. Upon removal of the inner catheter 34, the distal tip of the elastomeric properties of the distal soft tip jacket 174 of the outer catheter 36 permit the tapered portion 178 to return to its original internal diameter (0.045″).

In any of these examples, the inner and outer members (e.g., the inner tubular member and outer delivery sheath) may be configured so that when the engagement protrusion (e.g., ring) on the inner member is engaged with the releasable lock within the middle junction region of the outer member, the outer surface of the tapered distal tip of the outer member is substantially flush with a surface of the interface region of the inner member, as shown in FIGS. 7 and 8A-8B.

In the disengaged mode of operation, said inner diameter of the wall 180 of the tapered outer tip 164 of the outer member 36 is smaller than the outer diameter of the inner member 34. In the engaged mode of operation, the tapered outer tip 164 of the outer member 36 and the inner member 34 interact such that a dimensional transition between the outer diameter of the tapered outer tip 164 of the sheath lumen 120 and the outer diameter of the distal portion of the inner member 34 forms a substantially flush interface transition therebetween.

Referring further to FIGS. 9A-9D, the tapered portion 178 is contemplated in several embodiments of the expandable tapered designs. As shown in FIG. 9A, the elasticity of the outer catheter 36 at its distal tapered portion 178 is augmented by an expandable split ring 190 affixed at the tapered portion 178 which allows the distal outer tip 164 to expand (when interfaced with the inner catheter 34). The expandable split ring 190 has a slit 192 which allows the ring 190 to expand and contract, depending on the interference between the inner and outer catheters at their distal ends. This structure provides an additional reinforcement to prevent a permanent deformation of the tapered portion 178 during the inner catheter 34 removal and delivery of the stent (or the balloon).

Referring to FIG. 9B, in an alternative embodiment of the outer catheter 36, the tapered portion 178 may be configured with an expandable tip scaffold 194 which may be fabricated from NiTi wire and configured with a distal end 196 and a proximal end 198 which has a diameter larger than the diameter of the distal end 196. Due to its flexibility, the expandable scaffold 194, expands and contracts, when needed, and provides additional support to resist a permanent deformation of the jacket 174 at the tapered portion 178 during the inner catheter removal and delivery of the stent or the balloon member.

Another alternative embodiment of the tapered portion 178 at the distal end of the sheath 120 is shown in FIGS. 9C-9D, where wall 180 of the tapered portion 178 is shaped with slits 200 which extend longitudinally along the length of the tapered portion 178 spread apart along its perimeter. When the tapered portion 178 interfaces with the distal end of the inner member 34, the slits 200 temporarily widen to embrace the distal tip 162 of the inner catheter 34. This design can prevent a permanent deformation of the jacket 174 at its tapered portion 178 which may be caused by the inner catheter 34 removal or during the stent/balloon number delivery.

An important “seamless” aspect of the subject system is that for a transition between the outer diameter of the outer tip 164 of the sheath 120 (at the tapered portion 178 thereof) and the outer diameter of the distal tip 162 of the inner member 34 forms substantially gradual (smooth) transition therebetween.

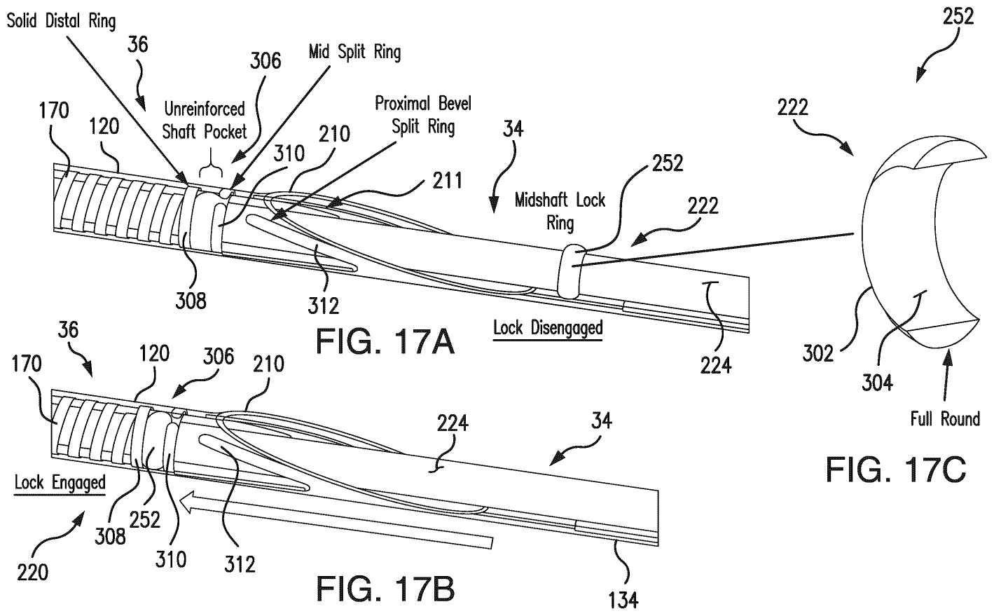

As shown in FIGS. 2C, 10A-10G, 11A-11C, 12A-12C, 13A-13B, 14A-14B, and 15A-15D, the subject system is built, at the middle section 42, with an interconnection mechanism 220 which includes the proximal coupler 130 formed at the proximal end 132 of the sheath 120 of the outer member 36, and a cooperating mechanism 222 formed at the outer surface of the inner member 34 (as depicted in FIGS. 17A-17B, 18B, 19A-19B, and 20A-20C).

The subject guide catheter extension/pre-dilatation system 10 may operate in an inner/outer catheters engagement mode and in an inner/outer catheters disengagement mode, which is accomplished by controlling the interconnection mechanism 220. The subject interconnection mechanism 220 is configured to engage/disengage the inner and outer catheters 34, 36 (as required by the cardiac procedure), as well as to prevent an unwanted forward displacement of the inner member 34 inside the outer delivery sheath 120. The engagement mode of operation allows the enhanced “pushability” of the “system as a whole” (with the outer catheter 36 connected and locked to the inner catheter 34) even with the connected pushing/pulling element 134 of the outer member 36 configured as a low profile and flexible element (as flexible or more flexible than the outer tubular sheath 120 of the outer catheter 36).

The interconnection unit 220 operates based on the interference between the proximal coupler 130 configured at the proximal end 132 of the sheath 120 and the cooperating mechanism 222 configured at the outer surface 224 of the inner member 34 when the inner surface 152 of the tubular body 150 of the sheath 120 (at its proximal end 132) engages the outer surface 224 of the cooperating mechanism 222 (on the inner member 34).

As an example, a number of interconnection mechanisms are envisioned to be applicable in the subject guide catheter extension/pre-dilatation system 10. The subject engagement mechanism is configured for controllable engagement/disengagement between the inner member 34 and the outer member 36, as well as to prevent a forward motion of the inner member 34 relative the outer delivery sheath 120 beyond a predetermined position.

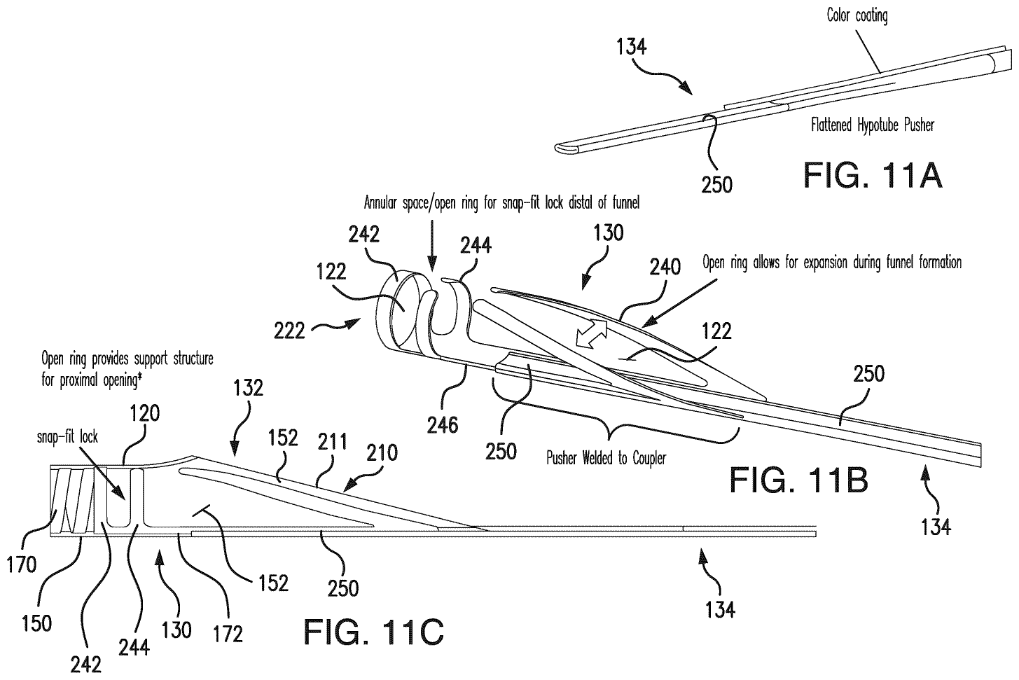

For example, as depicted in FIGS. 11A-11C, a laser cut coupler 130 may be configured with a proximal open (split) ring 240 and a pair of distal rings including a solid distal ring 242 and an open (split) distal ring 244. The proximal open ring 240, as well as the distal rings 242 and 244, is formed integrally with a coupler base 246. The coupler 130 may be formed from stainless steel or heat set NiTi. The pusher/puller element 134 of the outer catheter 36 and the mid-shift coupler (also referred to herein as a proximal coupler) 130 may be made from a memory metal (such as, for example, nitinol) so as to prevent deformation during antegrade or retrograde movement of the outer member and to prevent any deformation of the mid shaft coupler 130 during the stent (or other device) passage through the mid shaft portion of the outer catheter 36.

The open ring 240 is correlated with the proximal entry opening, (for example funnel shaped) 211 of the outer catheter 36 (shown in FIGS. 10A, 10D-10E and 11C). The proximal open ring 240 allows for expansion of the entrance 211 into the funnel 210 as needed for entrance/removal of the inner catheter 34 as required by the surgical procedure. As shown in FIGS. 10A, 10D-10E, and 11A-11C, the proximal open ring 240 provides a support for the proximal opening 210 of the funnel shaped proximal end of the sheet 120. The proximal ring 240 reinforces the entrance opening (“mouth”) 211 and prevents from the damage or a permanent deformation of the entrance opening, thus supporting elastic properties of the sheath 120 at the entrance opening 210. The distal rings 242, 244 create a snap-fit lock mechanism separate from the funnel's proximal open ring 240. The distal ring 242 is not expanded (being of a closed circular contour), while the opening of the split ring 244 expands during the displacement of the inner catheter 34 relative to the proximal coupler 130 of the outer catheter 36.

The base 246 of the coupler 130, as shown in FIGS. 11B-11C, may be flat, or preferably, is slightly arcuated (in the cross-section) to be congruent with the cooperating distal end 250 of the pusher 134 which has either flat or crescent (in crossing direction) contour. The pusher 134 may be fabricated from stainless steel or NiTi. The distal end 250 of the pusher 134 is welded (glued, adhered or otherwise affixed) to the base member 246 of the coupler 130. The PTFE liner (also shown in FIG. 7) 172 may encapsulate the coupler 130 as shown in FIG. 11C.

The sheath 120 is positioned in surrounding relationship with the coupler and the PTFE liner 172. The Pebax encapsulation, similar to the distal soft tip jacket 174, at the distal end 160 of the sheath 120 (shown in FIG. 7) may be used at the proximal end 132 of the sheath 120. The catheter shaft coil reinforcement 170 (also shown in FIG. 7) at the distal end of the outer catheter 36 can extend the length thereof to the proximal end of the outer catheter 36. As shown in FIGS. 11A-11C, and 17A-17C, and 18A-18B,

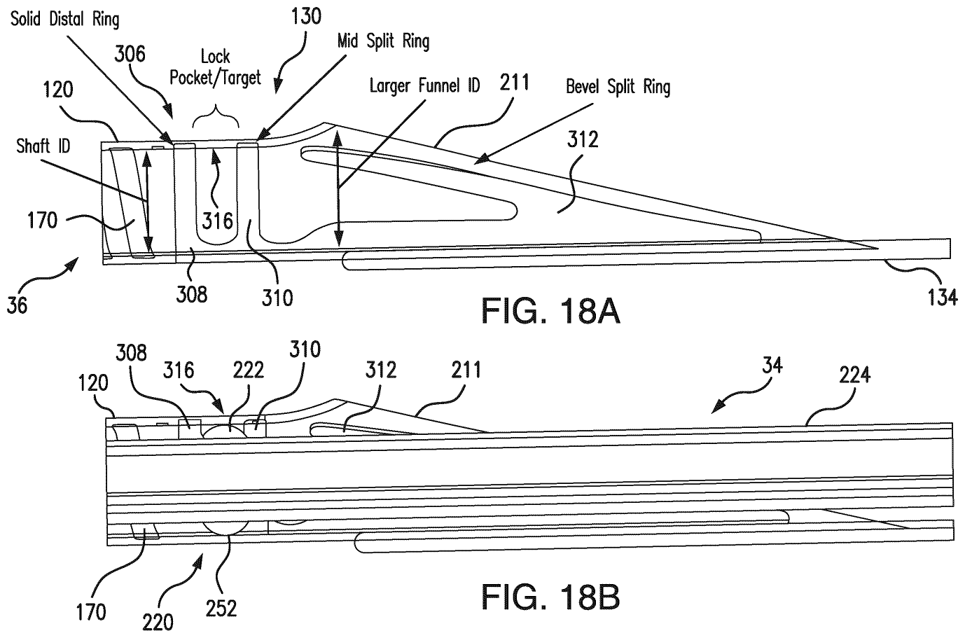

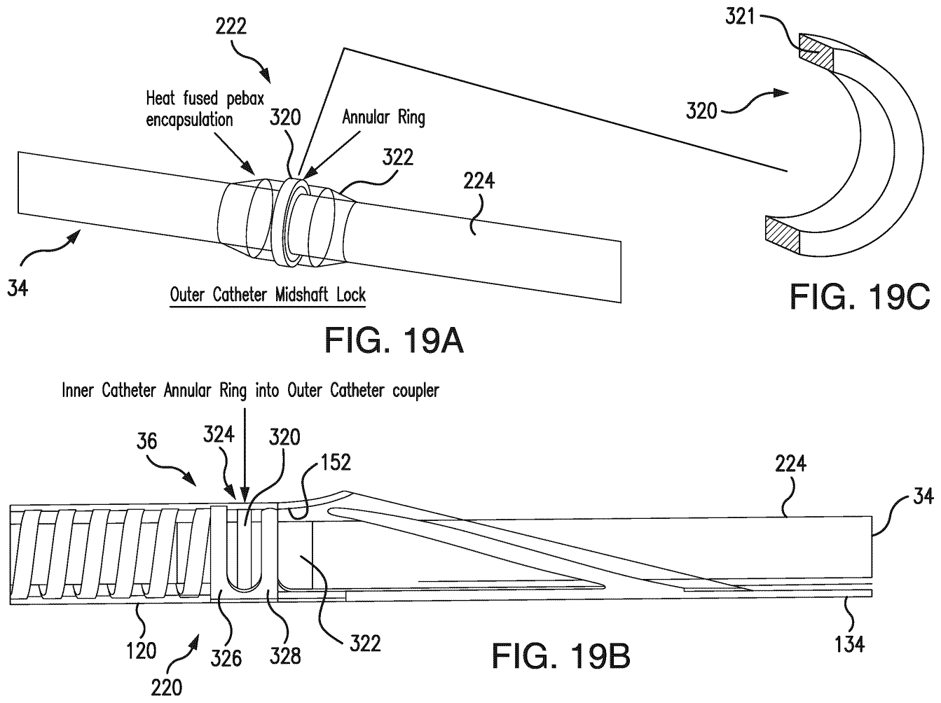

cooperating mechanism 222 for the specific embodiment shown in FIGS. 11A-11C further includes a mid-shaft lock ring 252 (shown in FIGS. 17B-17C and 18B) for the snap-fit locking. This lock ring is just one example of an engagement protrusion that extends from the outer surface 224 of the inner tubular shaft. FIG. 19A shows another example of an engagement protrusion. An engagement protrusion may one or more bumps. An engagement protrusion may reliably lock and engage in the middle junction of the outer delivery sheath between the tubular distal region and the proximal wire, as shown in FIG. 17A-17B.

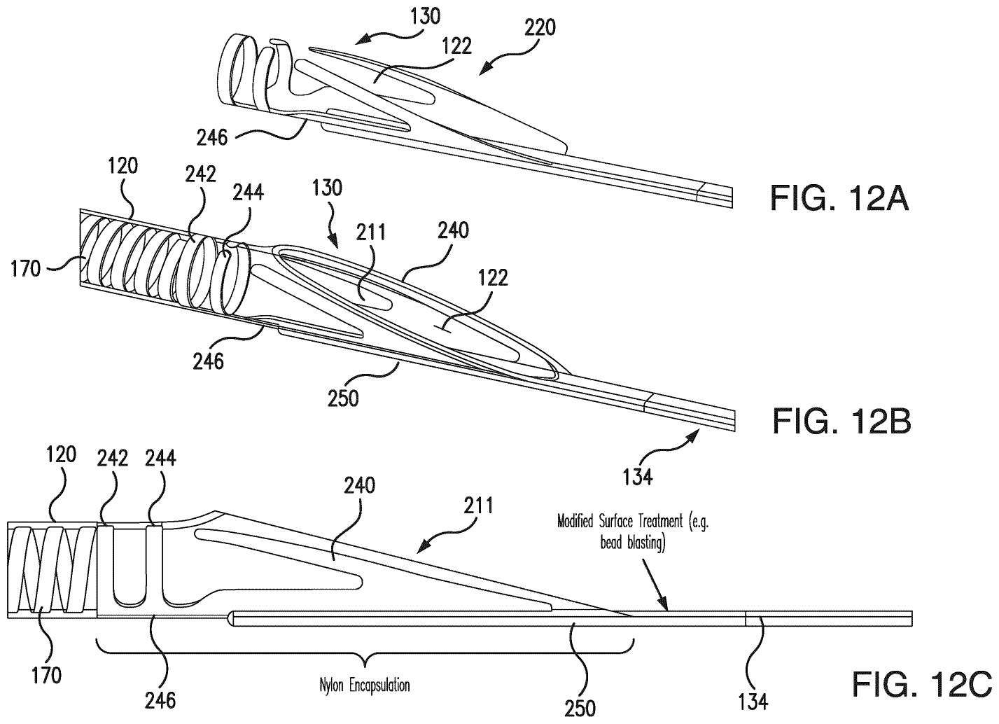

Another embodiment of the outer catheter's proximal entry structure shown in FIGS. 12A-12C, is similar to the one shown in FIGS. 11A-11C with certain modifications, including:
(a) an added thickness and additional material around the base 246 of the coupler 130;
(b) modified surface treatment (e.g., bead blasting) for improving the polymer encapsulation adhesion; and
(c) using the hard polymer (such as Nylon) encapsulation to provide additional support to the funnel to prevent damage which may impede the stent passage.

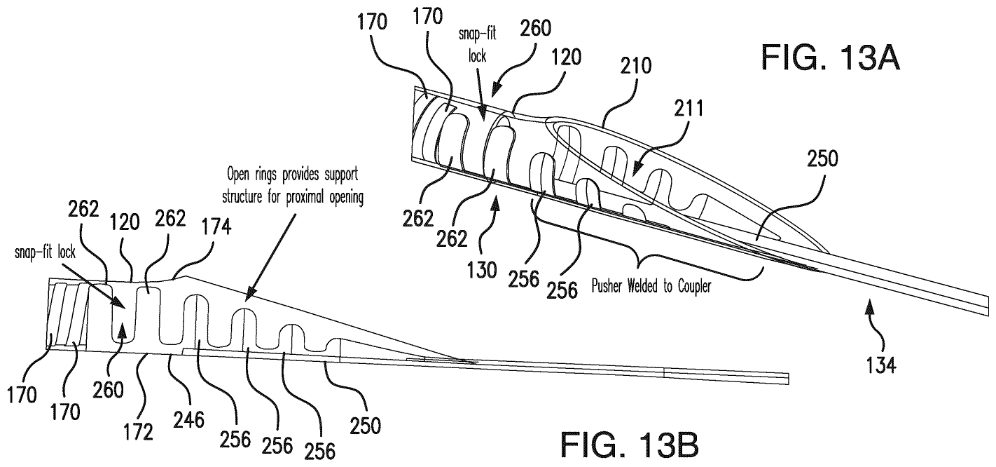

An additional embodiment of the coupler 130 at the proximal entry 210 (shown in FIGS. 13A-13B) features open rings (ribs) 256 which reinforce the entrance port 210. The snap-fit lock 260 is represented by at least two open rings 262 at the distal end of the coupler 130. The coupler 130, as shown in the modification presented in FIGS. 13A-13B, is preferably a laser cut coupler formed either from a stainless steel or heat set NiTi.

The hypo-tube pusher/puller 134 may be flattened at its distal end 250 and is welded to the base 246 of the coupler 130. The PTFE liner 172 extends underneath the coupler 130, and the Pebax encapsulation 174 envelopes the coupler 130 with the pusher 134 affixed thereto. The catheter shaft coil reinforcement structure 170 extends along the shaft 120 of the outer catheter 36 from the distal to the proximal end thereof. The snap-fit lock 260 cooperates with the round ring embodiment of the cooperating mechanism 222 shown in FIGS. 17A-17C and 18B. In some embodiments, the encapsulation 174 and/or the pusher/puller 134 may be color coated with a distinct color, as shown in FIG. 11A, to distinguish the outer member's pusher/puller 134 from other elements of the subject arrangement for the surgeon convenience and safety of the procedure.

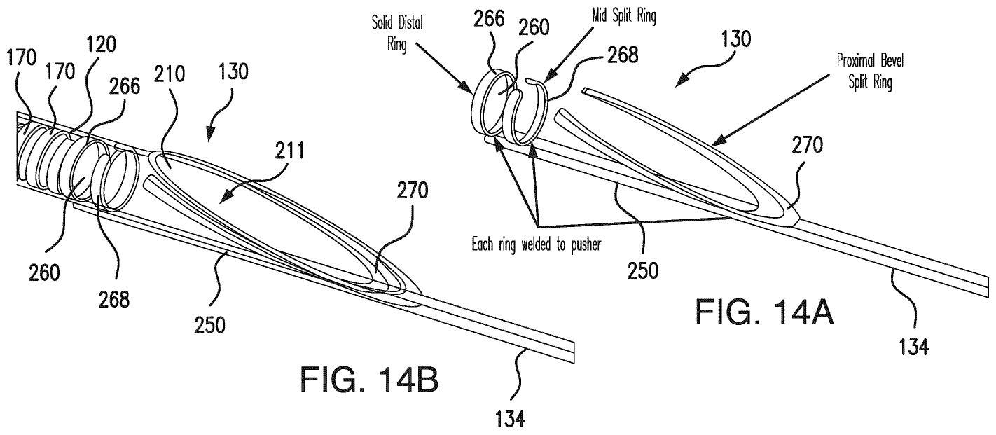

An additional modification of the coupler 130 is presented in FIGS. 14A-14B where the coupler 130 has individual rings 266, 268 welded to the distal end 250 of the pusher 134. As shown, the locking mechanism 260 is formed by the solid distal ring 266, and the mid split ring 268, with each ring 266, 268 welded to the pusher 134. The proximal bevel split ring 270 is also welded to the pusher 134. This design offers an increased flexibility in terms of the size and configuration of each ring 266, 268 and 270, and supports the formation of different funnel shapes/dimensions, as opposed to the laser cut coupler limited to a single diameter.

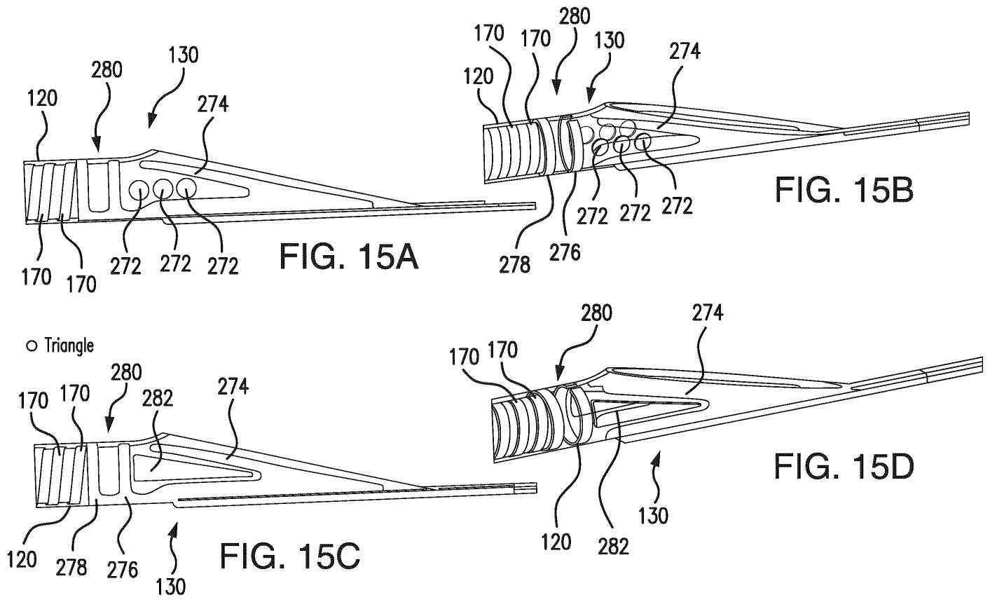

FIGS. 15A-15B depict another modification of the proximal coupler 130 which features funnel fenestrations which improve the contrast infusion flow rate by providing an additional open cross-sectional path for the fluid flow. As shown in FIGS. 15A-15B, circular openings 272 are formed in the sheath 120. The openings 272 are positioned in a predetermined pattern in a non-obstructive fashion with the proximal split ring 274 and the distal rings 276, 278 of the snap-fit lock structure 280. Shown in FIGS. 15C-15D, the coupler 130 is formed with triangular openings 282 formed in the sheaths 120 in the non-obstructive fashion with the proximal ring 274 and distal rings 278, 276 of the snap-fit lock 280.

Although only circular and triangular openings 272, 282, respectively, are shown in FIGS. 15A-15D, other configurations of the cutouts in the plastic encapsulation are also contemplated in the subject structure to allow the passage of an injected contrast fluid through the cutouts.

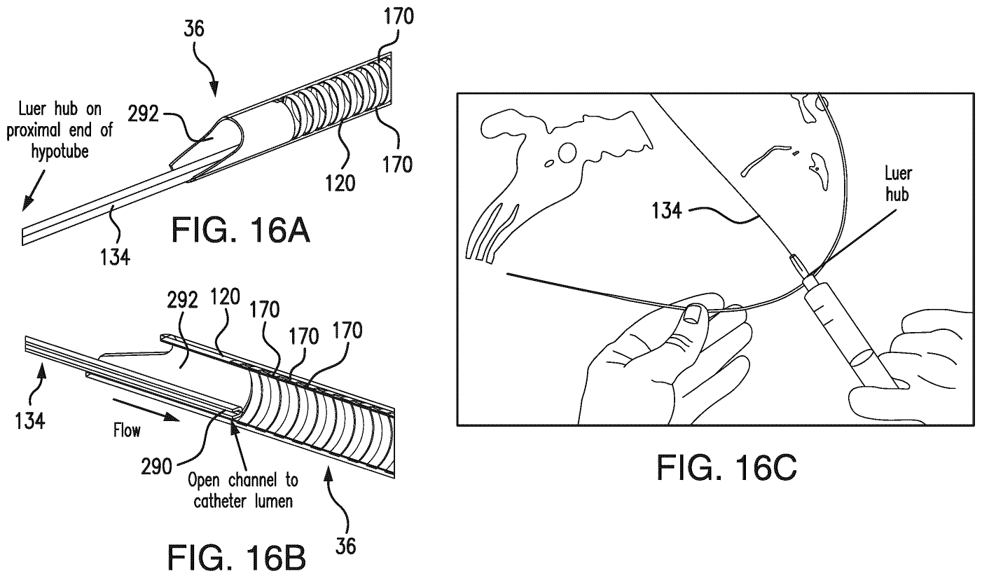

Referring to FIGS. 16A, 16B, and 16C, another embodiment of the proximal end of the outer catheter 36 is presented which is specifically designed as a potential solution to prevent an unwanted embolization situation when air is inadvertently entered with the fluid injected between the inner and outer catheters 34, 36. In order to prevent this, a flush lumen 290 is built into the pusher 134 via a flattened hypo-tube. A Luer hub is coupled to the proximal end of the hypo-tube (pusher 134) as shown in FIG. 16C, so that a surgeon can inject the fluid between the inner and outer catheters via the hypo-tube 134. When the fluid is entered into the outer catheter lumen 292 via the channel 290 in the hypo-tube 134, the entrance of air bubbles between the inner and outer catheters is prevented.

Further, referring to FIGS. 17A-17C, the interconnection unit 220 between the proximal coupler 130 presented in FIGS. 11A-11E, 12A-12C, 13A-13B, 14A-14B, and 15A-15D, includes the cooperating member 222 in the form of an annular round ring 252 (also referred to herein as a mid lock ring) formed on the outer surface 224 of the inner catheter 34. The stainless steel annular ring 252 is contoured with a full round surface which permits for smallest reversible engagement/disengagement from the need split-ring feature of the outer catheter coupler 130. The ring 252 as shown in FIG. 17C has a rounded contour on the outer surface 302 for smooth locking/unlocking action. The inner surface 304 of the ring 252 is also a smooth structure which is engaged with the outer surface 224 of the inner catheter 34.

FIG. 17A depicts the disengaged configuration of the inner catheter 34 relative to the outer catheter 36. FIG. 17B is representative of the lock engaged configuration when the inner catheter 34 is received and locked inside of the opening 210 at the proximal end of the sheath 120 so that the ring 252 is engaged in the snap-fit lock 306 formed by the distal solid ring 308 and the mid split ring 310. While in position, the proximal bevel split ring 312 encircles the inner catheter 34, and the ring 252 is locked in the snap-fit lock 306, thus engaging the inner and outer catheters for surgical manipulation as required by a surgical procedure.

During the longitudinal motion of the inner catheter 34 inside the outer catheter 36, while the ring 252 passes through the proximal bevel split ring 312 and the mid split ring 310, the arms of these rings are expanded from the original position to create a sufficient room for the ring 252 to pass. When in position, i.e., the ring 252 is received between the rings 308 and 310, the arms of the bevel split ring 312 and the split ring 310 return to their original closed position. The ring 252, being trapped between the rings 308, 310, is snap-fit locked therebetween, thus preventing the inner and outer catheters relative displacement.

Referring to FIGS. 18A-18B, detailing the structure shown in FIGS. 17A-17C, it is shown that the section (pocket) 316 of the sheath 120 of the outer catheter 36 is not reinforced by the coils 170 and deflects when the mid shaft lock ring 252 is inserted between the solid distal ring 308 and the mid split ring 310 of the snap-fit lock 306. The deflected portion 316 of the sheath 120 between the rings 308 and 310 provides additional retention force to maintain the inner and outer catheters 34, 36 in locking engagement.

The stainless steel annular ring 252 may be attached to the outer surface 224 of the inner catheter shaft 34 via an adhesive. The lock ring geometry (full round surface) allows for a smooth reversible engagement/disengagement from the laser cut features of the outer catheter's coupler 130. The distal ring 308 of the snap-fit lock 306 prevents further distal motion of the inner catheter 34, while the mid-split ring 310 opens upon contact with the mid shaft lock ring 252 and provides the tactile snap. The proximal bevel split ring 312 allows for the funnel 211 to be opened to an internal diameter larger than the internal diameter of the rest of the shaft 120. It also allows for a smooth passage of the mid-shaft lock ring 252.

The interference between the unreinforced shaft pocket 316 and the mid shaft lock ring 252 provides retention of the inner catheter 34 to the outer catheter 36 until the user is ready to remove the inner catheter 34 from the outer catheter 36, thus disengaging the snap fit lock therebetween. The force required to disengage the lock mechanism can be tailored from 0.1 to 2.0 lbs.

Referring to FIG. 19A-19C, another alternative embodiment of the mid shaft lock is presented which includes a square annular ring (formed of a metallic or a polymeric material) 320. Unlike the ring 252, shown in FIGS. 17A-17C and 18B, the ring 320 has a square cross-section 321, shown in FIG. 19C. The square annular ring 320 is affixed to the outer surface 224 of the inner catheter 34 with a heat fused Pebax encapsulation 322. Alternatively, it may be glued to the inner catheter outer surface 224. As shown in FIG. 19B, when the inner catheter is in locked position, the square annular ring 320 snaps into the snap-fit lock 324 formed by the solid ring 326 and split ring 328, with the encapsulation 322 in contact with the internal surface 152 of the sheath 120 and with the ring 320 positioned between the rings 326 and 328.

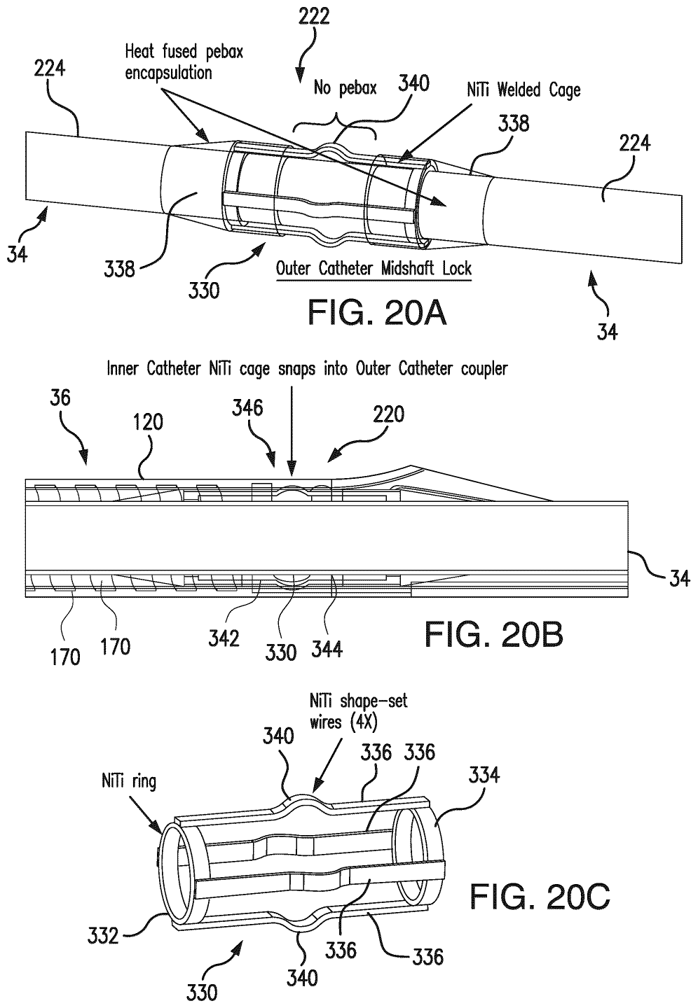

In a further alternative embodiment, shown in FIGS. 20A-20C, the mid-shaft lock mechanism 220 is formed with the cooperating member 222 in the form of a cage shaped structure 330 having two NiTi rings 332, 334 connected together through a number (for example, 4) of NiTi shape-set wires 336. As shown in FIG. 20A, the cage 330 is affixed to the outer surface 224 of the inner catheter 34 either by gluing or by heat fused Pebax encapsulation 338. Each of the wires 336 has an arcuated extending portion 340 which is left free from the encapsulation 338 as shown in FIGS. 20A and 20B.

As shown in FIG. 20B, for the locking configuration, the cage structure 330 snaps into the outer catheter's coupler 130. The un-encapsulated arcuated portion 340 of each wire 336 extends outside the encapsulation 338 and away from the wires 336 of the cage 330. When the cage 330 is received between the rings 342 and split ring 344 of the snap fit mechanism 346, the locking mechanism 346 is actuated, and the inner and outer catheters 34, 36 are engaged.

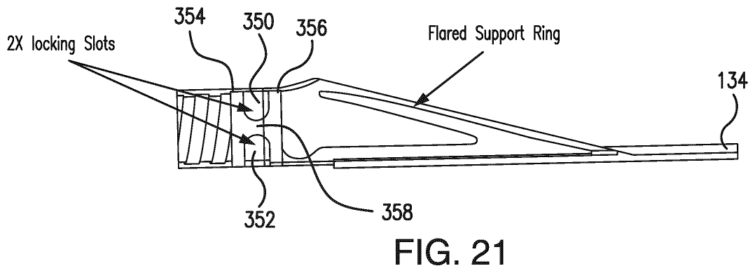

Referring further to FIG. 21, the proximal coupler 130 of the outer catheter 36 may include two locking slots 350, 352 which are formed by connected rings 354 and 356 with a connecting element 358.

Referring to FIGS. 17A-17C, 18A-18B, 19A-19B, 20A-20C, as well as 10A-10G, 11A-11C, 12A-12B, 13A-13B, 14A-14B, 15A-15D, and 21, when a surgeon linearly displaces the inner member 34 within the internal channel 122 of the proximal coupler 130, the snap-fit annular ring 252, 320, or the cage 330 enters the channel 122 between the arms of the proximal ring 240, 312, which are flexibly bent outwardly to permit forward motion of the inner catheter 34 (towards the distal tip 162). When the snap-fit annular ring 252, 320 or the cage 330 passes further through the mid split ring 244, 262, 268, 310, 328 of the snap-fit lock, the arms of the bevel proximal ring return to their original position, but the arms of the mid split ring are flexibly bent outwardly to allow the ring 252, 320 of the cage 330 to the position between the distal solid ring and the mid split ring. After the ring/cage 252, 320, 330 is snap-fit between the rings of the snap-fit locking mechanisms, the arms of the mid-split ring return to their original position.

In order to disengage the inner member 34 from the outer member 36, the surgeon pulls the inner member 34 from the internal channel of the proximal coupler 130. During the removal of the snap-fit annular ring/cage 252, 320, 330 from the channel, the pulling action causes the arms of the mid-split ring to bend outwardly to permit the passage of the snap-fit annular ring/cage 252, 320, 330 therebetween, thus freeing the inner catheter 34 from the proximal coupler 130 of the outer catheter 36.

Returning to FIG. 3D, the inflation lumen distal shaft 66 at the middle section 42 of the subject guide catheter/pre-dilatation extension system 10 may be manufactured with braid reinforcement structure 260. The braid reinforcement member 260 creates a somewhat flexible tubing connected to the cooperating mechanism 222 of the interconnection unit 220 of the inner member 34. The RX (Rapid Exchange) port 94 for passing the guide wire 12 may be formed through the wall of the braid reinforced inflation lumen distal shaft 66.

The braid reinforcement structure 260 may be configured with metallic patterns or wires within the braid reinforced inflation lumen distal shaft 66 to prevent kinking, which would give the shaft 66 a longitudinal stiffness. The metal braid 260 may be embedded in the braid reinforced shaft 66 to add increased flexibility thereto required for retraction of the inner member 34 relative to the outer delivery sheath 120 during the procedure.

A flat wire helical coil (made, for example, from a shape memory alloy, such as Nitinol) with a wire thickness of approximately 1 mil to 3 mils may be embedded in the braid 260. This coil may be formed with a very thin coating of plastic placed onto its inner and outer surfaces, which facilitates the reduction of the wall thickness of the inflation lumen distal shaft 66 to less than 7 mils and preferably to approximately 5 mils.

The principles of reinforcing the tubular members by the catheter shaft coil reinforcement 170 in a form of a flat wire helical coil 262 or forming the tubular members from the flat wire helical coil may be applied in the subject guide catheter extension/pre-dilatation system 10 to the outer delivery sheath 120 (as shown in FIGS. 7, 8B, 9A-9D, 10A, 11C, 12B-12C, 13A-13B, 14B, 15A-15C, 16A-16B, 17A-17B, 18A-18B, 19B, 20B, and 21, as well as to the micro-catheter 46 (as shown in FIGS. 2A-2B, 5A, 22, and 24A-24B). In the outer delivery sheath 120 and/or the micro-catheter 46, such flat wire helical coil may be embedded in predetermined positions along the length of the walls thereof, for example, at the proximal and or distal ends.

Alternatively, the entire length of the outer delivery sheath 120 and/or micro-catheter 46 may be formed with the flat wire helical coil. The pitch between the coils may be adjusted to provide the flexibility gradient along the length of the tubular member (sheath 120 and or micro-catheter 46) increasing towards the distal end thereof to facilitate atraumatic operation.

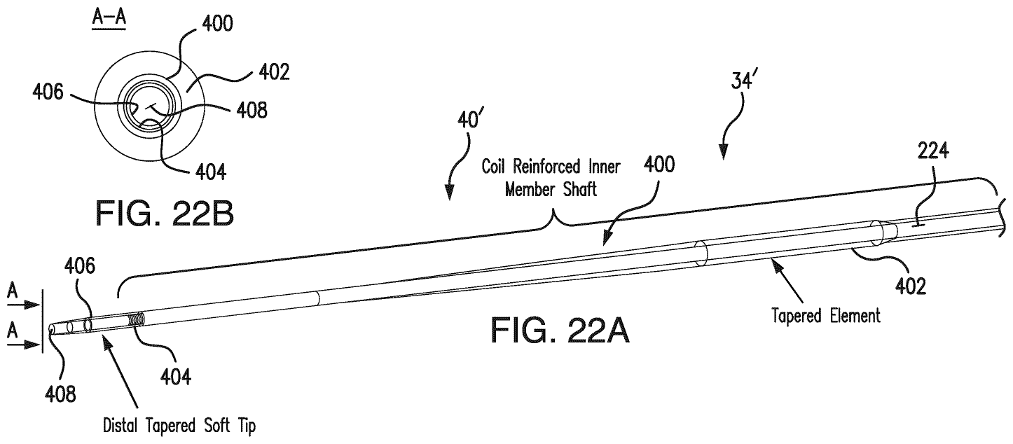

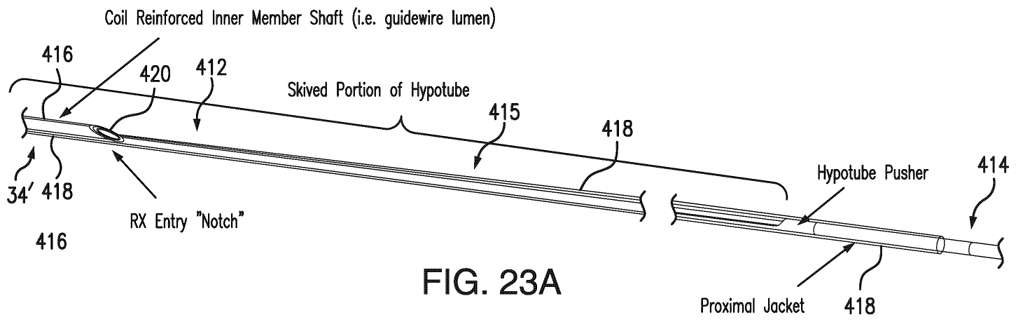

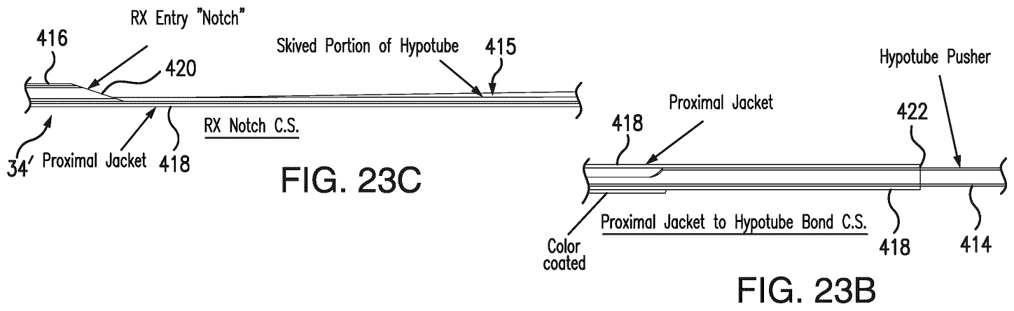

Referring to FIGS. 22A-22B and 23A-23C, rather than utilizing a standard over-the-wire (OTW) guidewire lumen, a monorail Rapid Exchange (RX) design of the inner catheter 34′ may be implemented to allow for the use of short guidewires. In the embodiment shown in FIGS. 22A and 22B, which represent the isometric view of the subject coil reinforced inner member shaft 400 and the side view taken along lines A-A thereof, the distal section 40′ of the inner member 34′ includes a tapered element 402 attached to the outer surface 224 of the inner member 34. The outer shaft 400 of the inner member 34′ is a coil reinforced with coil reinforcement structure 404 extending from the distal tip 406 to the RX entry port 94 shown in FIGS. 2A-2C and 3C-3D. The distal tip 406 is a tapered soft tip which, along with the tapered element 402, interfaces with inner surface of the outer catheter 36 when the inner catheter 34′ is charged in the outer catheter 36, as required by the surgical procedure.

The distal section 40′ contains a concentric guidewire lumen 408, which communicates with the RX entry port at the proximal end of the inner catheter 34 (shown in FIGS. 2A-2C, and 3C-3D).

Shown in FIGS. 23A-23C, the proximal end 412 of the monorail micro-catheter embodiment shown in FIGS. 22A-22B utilizes a skived hypo-tube pusher 414. The proximal end 412 of the coil reinforced inner member shaft 416 and the hypo-tube pusher 414 are encapsulated in the proximal outer jacket 418, which extends as a tube along the proximal end 412 of the monorail micro-catheter embodiment of the inner member 34′, from (and including) the coil reinforced inner member shaft 416 (which serves as a guidewire lumen) 408 shown in FIGS. 22A-22B and the hypo-tube pusher 414.

The embodiment depicted in FIGS. 23A-23B features an RX guidewire “notch” termination/entry 420 which is fabricated by piercing the proximal outer jacket 418. Subsequently, the coil reinforced inner member shaft 416 is inserted into the proximal outer jacket tube 418 via the RX entry “notch” 420. The skived hypo-tube 415 is further inserted into the proximal outer jacket tube 418 via its lumen 422, and the polymers of the coil reinforced inner member shaft 416 and the proximal outer jacket tube 418 are fused together to connect the inner member shaft 416 and the pusher 414 and, thus, to form the proximal end 412 of the monorail micro catheter inner member 34′.

For convenience of the surgeon, the pushing/pulling element 134 of the outer catheter 36 may be colored (color coated), as shown in FIG. 11A, to have a distinguished color to differentiate it from other elements of the system, such as the pushing/pulling element of the inner catheter 34, as well as from the usual gray or silver color of a coronary guidewire used to deliver the device or a stent delivery system. Alternatively, the proximal outer jacket 418 of the pushing-pulling element 414 may be color coated to distinguish its color from the colors of other elements in the subject system.

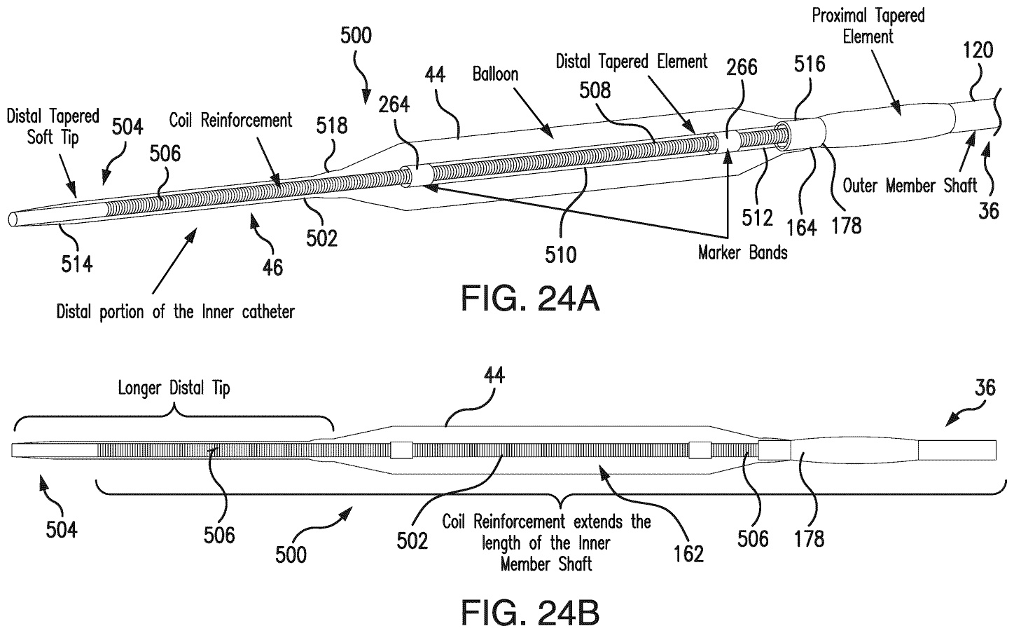

Referring further to FIGS. 24A-24B, representative of an additional coil reinforced balloon catheter embodiment 500 of the inner catheter, the structure combines the reinforced shaft properties of the micro-catheter 46 with that of the dilatation balloon 44 with the following attributes:
a. Coil reinforced shaft 502 provides an additional kink resistance and pushability while still maintaining flexibility for navigating tortuous vasculature; and
b. The structure's longer distal tip 504 contains low profile, tapered soft tip to facilitate crossing the stenosis and tight lesions.

As shown in FIGS. 24A-24B, the distal section 504 of the subject structure 500 includes the inner member shaft 500 reinforced with the coil reinforcement structure 506 which extends the length of the inner member's shaft 500. The distal tapered element 508 is positioned on the inner member shaft 500 and extends between the ends 510 and 512 in encircling relationship with the inner member shaft 500. The distal tapered soft tip 514 may be in the form of the micro catheter 46 which is positioned at the end of the coil reinforced shaft 500.

Similar to the embodiment presented in FIGS. 22A-22B, the balloon member 44 is positioned on the inner member shaft 500 with the radio opaque markers 264 and 266 positioned on the inner member shaft 500 within the balloon member 44. At its proximal end 516, the balloon member 44 interferes with the outer tip 164 of the proximal tapered element 178 of the outer member sheath 120. At the distal end 518, the balloon member 44 snugly embraces the shaft 500.

Returning to FIGS. 1-24B, in operation, for performing the cardiac procedure, and specifically the pre-dilatation routine, a proximal end of the coronary guidewire 12 is entered into the RX port 94 formed in the inflation lumen distal shaft 66, and is extended through the inner channel (GW lumen 96) of the inner member 34 towards and beyond the outermost distal end 52 of the micro-catheter 46. Subsequent thereto, the guide catheter 14 is advanced into the blood vessel 16 of interest.

Subsequently, the outer delivery sheath 120 of the outer member 36 locked with the inner member 34 therewithin, are placed first with the micro-catheter 46 in the internal channel 48 of the guide catheter 14, and both inner and outer members 34, 36 as a single unit, are integrally advanced within the guide catheter 14 towards the treatment site 22. The outer member's sheath 120 and the inner member 34 may be integrally displaced by pushing the outer member pusher 134. This action causes the micro-catheter 46 of the inner member 34 to slide along the GW 12 along with the outer member 36 until they extend beyond the distal end 50 of the guide catheter 14, and reach the lesion site 92. In this step of the procedure, the balloon member 44 is in its deflated configuration.

The guidewire 12 which extends beyond the distal end 50 of the guide catheter 14, serves as a guide along which the micro-catheter 46 (with the deflated balloon 44 attached to the distal tip 162) slides towards the treatment site 26.

Subsequently, the balloon member 44 (which is positioned at the treatment site 22) is inflated by the balloon inflation system 62 connected to the inflation hub 56 through the inflation lumen formed by the inflation lumen distal shaft 66 and the inflation lumen hypotube 64 in order to compress the plaque and to widen the blood passage inside the blood vessel 16.

Subsequently, once the lesion has been dilated, the balloon 44 is deflated, and the outer delivery sheath 120 may be advanced across the lesion 22 either as an integral unit with the inner member 34 (in the engaged mode of operation), and the inner member may be subsequently disengaged (unlocked) from the outer delivery sheath 120 and removed from the sheath 120.

Alternatively, the inner member 34 may be disengaged and withdrawn from the sheath 120 directly after the lesion dilatation, while the outer member 36 is advanced across the lesion 22.

The sheath 120 may be left in place (directly after the dilatation of the lesion) proximal to the treatment site.

Subsequent to pulling the inner member 34, the stent can be delivered to the site 22. The stent, in its closed configuration, may be introduced into the blood vessel 16 inside the sheath 120. When in place, the stent supporting balloon (not shown) may be expanded, thus opening the stent. Subsequently, the outer delivery sheath 120 is removed, leaving the opened stent in the blood vessel 16.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

In general, as described herein, the term machine learning and “machine learning agent” refers to a class of computational techniques that enable systems to learn patterns and make decisions or predictions based on data, without being explicitly programmed for each specific task. Machine learning involves the development of algorithms that can generalize from examples, adapt to new inputs, and improve performance over time. A machine learning agent is any system or component (e.g., subsystem) that employs such algorithms to perform tasks such as classification, regression, clustering, decision-making, or control. These machine learning agents may operate autonomously or as part of a larger system, and they can be implemented in software, hardware, firmware or a combination of any of these.

Machine learning encompasses a broad range of methodologies, which can be broadly categorized into supervised learning, unsupervised learning, semi-supervised learning, and reinforcement learning, any of which may be used by the machine learning agents described herein. In supervised learning, the agent is trained on labeled data, learning to map inputs to known outputs. Unsupervised learning involves discovering hidden patterns or structures in unlabeled data, such as clustering or dimensionality reduction. Semi-supervised learning combines both labeled and unlabeled data to improve learning efficiency. Reinforcement learning, on the other hand, involves an agent interacting with an environment and learning to make decisions by receiving feedback in the form of rewards or penalties.

Additionally, machine learning includes specialized subfields such as deep learning, which uses multi-layered neural networks to model complex relationships in data, and transfer learning, where knowledge gained from one task is applied to a different but related task. Other techniques include ensemble learning, which combines multiple models to improve predictive performance, and online learning, where models are updated continuously as new data arrives. These approaches may be used individually or in combination, depending on the requirements of the invention.

In the context of the methods and apparatuses described herein, the term “machine learning” and “machine learning agent” are intended to be interpreted broadly, encompassing any computational method or system that utilizes data-driven learning to perform tasks, adapt behavior, or improve outcomes. This includes but is not limited to neural networks, decision trees, support vector machines, probabilistic models, and evolutionary algorithms. The invention may incorporate one or more of these techniques to achieve its objectives, and the scope of the patent should be understood to include all such variations and implementations.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

“An embodiment,” “in an embodiment”, “in another embodiment,” “embodiments,” “certain embodiments,” or “in some embodiments” each has the meaning herein of “in one or more embodiments”. Similarly, “a configuration,” “in a configuration,” in one configuration,” “in another configuration,” “configurations,” “certain configurations,” or “in some configurations” each has the meaning herein of “in one or more configurations.”

The terms “e.g.,” “such as,” “for example,” “and so forth,” and “etc.” mean that what follows the “e.g.,” “such as,” or “for example” or what precedes the “etc.” or “and so forth,” is a list of examples and there may be other items that could also serve as examples but are not listed.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system, the system comprising:
an outer delivery sheath having a proximal region, a distal region, and a middle junction between the proximal and distal regions, wherein the middle junction comprises a releasable lock; and
an inner tubular shaft having a proximal end region, a distal end region, and an engagement protrusion extending radially from an outer surface of the inner tubular shaft, wherein the engagement protrusion is configured to engage the releasable lock when a distal end region of the inner tubular shaft extends distally out of a distal end of the outer delivery sheath;
wherein the engagement protrusion comprises a contact surface configured to drive advancement of the outer delivery sheath by pushing from the middle junction of the outer delivery sheath.

2. The system of claim 1, wherein the inner tubular shaft comprises a contact region proximal to a distal end of the inner tubular shaft that is configured to form a substantially flush interface with the distal end of the outer delivery sheath when the engagement protrusion engages the releasable lock.

3. The system of claim 1, further comprising a connector configured to releasably connect the proximal end region of the outer delivery sheath to the proximal end region of the inner tubular shaft when the engagement protrusion engages the releasable lock.

4. The system of claim 1, wherein the releasable lock comprises a friction lock.

5. The system of claim 1, wherein the releasable lock comprises an engagement slot on the outer delivery sheath configured to receive the engagement protrusion from the inner tubular shaft.

6. The system of claim 1, wherein the releasable lock comprises a lock ring configured to elastically expand.

7. The system of claim 1, wherein the engagement protrusion comprises a ring.

8. The system of claim 1, wherein the proximal region of the outer delivery sheath comprises a wire.

9. The system of claim 1, wherein the distal region of the outer delivery sheath comprises a tubular distal region.

10. The system of claim 1, wherein the inner tubular shaft comprises a rapid exchange notch having an opening into a lumen extending from the rapid exchange notch to a distal end of the inner tubular shaft.

11. The system of claim 1, wherein the inner tubular shaft is configured to extend more than 1.5 cm distally out of a distal end of the outer delivery sheath when the engagement protrusion engages the releasable lock.

12. A system, the system comprising:
an outer delivery sheath having a proximal wire, a tubular distal region, and a middle junction between the proximal wire and the tubular distal region, wherein the middle junction comprises a releasable lock; and
an inner tubular shaft having a proximal end region, a distal end region, and an engagement protrusion extending radially from an outer surface of the inner tubular shaft, wherein the engagement protrusion is configured to lock with the releasable lock when a distal end region of the inner tubular shaft extends distally out of a distal end of the tubular outer delivery sheath;
wherein the distal end of the outer delivery sheath is configured to from a flush interface with a contact region of the inner tubular shaft that is proximal to a distal end of the inner tubular shaft when the engagement protrusion engages the releasable lock;
further wherein the engagement protrusion comprises a contact surface configured to drive advancement of the outer delivery sheath by pushing the middle junction.

13. The system of claim 12, further comprising a snap connector configured to releasably secure a proximal end region of the outer delivery sheath together with the proximal end region of the inner tubular shaft when the engagement protrusion is locked with the releasable lock.

14. The system of claim 12, wherein the releasable lock comprises a friction lock.

15. The system of claim 12, wherein the releasable lock comprises an engagement slot on the outer delivery sheath configured to receive the engagement protrusion from the inner tubular shaft.

16. The system of claim 12, wherein the releasable lock comprises a lock ring.

17. The system of claim 12, wherein the engagement protrusion comprises a ring.

18. The system of claim 12, wherein the inner tubular shaft comprises a rapid exchange notch having an opening into a lumen extending from the rapid exchange notch to a distal end of the inner tubular shaft.

19. The system of claim 12, wherein the inner tubular shaft is configured to extend more than 1.5 cm distally out of a distal end of the outer delivery sheath when the engagement protrusion is locked with the releasable lock.

20. A system, the system comprising:
an outer delivery sheath having a proximal wire, a tubular distal region, and a middle junction between the proximal wire and the tubular distal region forming an opening into a lumen of the tubular distal region, wherein the middle junction comprises a releasable engagement region;
an inner tubular shaft having a proximal end region, a distal end region, and an engagement protrusion extending radially from an outer surface of the inner tubular shaft, wherein the engagement protrusion is configured to engage with the releasable engagement region when a distal end region of the inner tubular shaft extends distally out of a distal end of the tubular outer delivery sheath;
a connector configured to releasably secure a proximal end region of the outer delivery sheath together with the proximal end region of the inner tubular shaft when the engagement protrusion is engaged with the releasable engagement region;
further wherein the engagement protrusion comprises a contact surface configured to drive advancement of the outer delivery sheath by pushing the middle junction.
